(19) United States
(12) Patent Application Publication
(10) Pub. No.: US 2005/0222039 A1
(43) Pub. Date: Oct. 6, 2005

(54) Animal treatment

(76) Inventors: Renata Montenegro-Lohr, Hamilton (NZ); Allan Nixon, Tamahere (NZ); Allan Pearson, Hamilton (NZ); Tanya Soboleva, Hillcrest, Hamilton (NZ)

Correspondence Address: Abelman Frayne & Schwab, 150 East 42nd Street, New York, NY, 10017-5612, US

(21) Appl. No.: 10/496,723

(22) PCT Filed: Nov. 25, 2002
(86) PCT No.: PCT/NZ02/00259

§ 371 Date: Feb. 24, 2005

(30) Foreign Application Priority Data
Nov. 23, 2001 (NZ) 515178

(51) Int. Cl. A01K 67/00; A61K 39/395

(52) U.S. Cl. 514/15; 424/145.1; 800/8

(57) ABSTRACT

A method for affecting a physiological response of an animal to circulating level of prolactin and/or prolactin mimetics, characterised by the step of a) modulating prolactin receptors.

### TECHNICAL FIELD

The present invention relates to a method of animal treatment.

### BACKGROUND ART

The understanding of an animal's physiological processes has long been a goal of researchers and the agricultural industry.

A better understanding allows the development of better agricultural practices, improving productivity, farm management and profitability.

With a greater understanding comes the ability to manipulate an animal's physiological processes, further boosting productivity and profitability.

Prolactin (PRL), a hormone of the anterior pituitary whose secretion varies seasonally in many species (Table 1), is involved in the physiological regulation of growth and development, hair and wool growth, reproduction, water and electrolyte balance, metabolism, behaviour and immune function [Bole-Feysot et al., 1998, Goffin et al., 2002].

For example, prolactin has previously been implicated in the control of hair growth in various species [Lincoln, 1989] including wool growth cycles in primitive and shedding breeds of sheep [Lincoln, 1990; Lincoln and Ebling, 1985].

More recently, prolactin receptors (PRLR) have been identified in the wool follicle [Choy et al., 1997] revealing a physiological mechanism whereby circulating prolactin can mediate wool growth cycles. However, to date the effects of prolactin on wool and hair growth in seasonal mammals, and particularly in modern non-shedding sheep breeds, are not well understood.

It would be desirable to more fully understand this relationship and to provide methods of manipulating prolactin dependent processes including, but not limited to, lactation, fertility and meat yields.

Prolactin is a peptide hormone comprising approximately 200 amino acids and a molecular weight of 23,000 kDa [Freeman et al., 2000]. Circulating prolactin is synthesised and released by specialised pituitary cells called lactotrophs, under the control of hypothalamic factors [Freeman et al., 2000].

Prolactin is also reported to be synthesised in an increasing number of extra-pituitary tissues allowing for local autocrine and paracrine effects [Wu et al., 1995; Craven et al., 2001]. It is thought to be responsible for as many as 300 different effects on central and peripheral tissues [Bole-Feysot et al., 1998; Goffin et al., 2002].

Prolactin Secretion

Pituitary prolactin secretion is influenced by physiological factors including photoperiod, temperature, pregnancy, parturition and lactation and stress.

Photoperiod: Prolactin secretion varies seasonally in many species (Table 1) being higher in long days (summer) than in short days (winter).

Temperature: A direct effect of the ambient temperature on prolactin concentration has been observed in cattle [Wettermann and Tucker, 1974; Wettermann et al., 1982] and goats [Prandi et al., 1988] with increasing prolactin secretion with rising temperatures.

Pregnancy and lactation: During pregnancy the maternal pituitary increases in size, primarily as a result of hyperplasia and hypertrophy of lactotrophic cells [Djiane and Kelly, 1993]. Maternal plasma prolactin levels remain low throughout most of the gestational period but increase rapidly in late term to reach maximal levels around the time of parturition.

In sheep, most studies [Fitzgerald et al., 1981; Kelly et al., 1974; Kendall, 1999; Lamming et al., 1974] show that maternal prolactin concentrations range between 10 ng/mL and 50 ng/mL during the first 100 days of gestation. These levels are comparable to or lower than basal prolactin concentrations in non-pregnant ewes over the same period [Fitzgerald et al., 1981, Kendall, 1999).

A rise in maternal prolactin concentration is usually observed a few days before parturition [Kelly et al., 1974; Lamming et al., 1974, Kendall, 1999]. During the final stages of labour and at parturition, rapid pulses of prolactin are released, levels reaching between approximately 100 ng/mL and 700 ng/mL [Kelly et al., 1974; Kendall, 1999; Lamming et al., 1974; Peterson et al., 1990].

Lactation is also associated with raised plasma prolactin. In sheep, basal prolactin concentrations throughout early lactation range from 100-150 ng/mL, however suckling causes a rise in prolactin levels to as high as 800 ng/mL [Kendall, 1999; Lamming et al., 1974].

By mid-lactation, basal levels are 20-100 ng/mL and suckling causes a rise in prolactin concentration from 20 ng/mL to up to 400 ng/mL. Plasma prolactin concentrations decline as lactation advances [Kendall, 1999; Lamming et al., 1974] and declines further after weaning [Rhind et al., 1980].

Prolactin Receptors

Prolactin has been shown to bind to specific high affinity cell surface receptors.

Signal transduction via these receptors initiates a cascade of tissue-specific gene transcription and translation resulting in physiological adaptations [Freeman et al., 2000].

Prolactin receptors have been identified in wide variety of tissues including the liver, uterus, mammary gland, kidney and skin [Barash et al., 1983; Cassy et al., 1999; Choy et al., 1997].

Multiple forms of prolactin receptor can arise by alternative splicing of a single gene [Ormandy et al., 1998; Bole-Feysot et al., 1998]. Two types of mRNA encoding a long and a short form of the prolactin receptor are detected in ovine and bovine tissues [Anthony et al., 1995]. While the specific functions of the variant proteins are uncertain it is presently thought that the major physiological effects of prolactin are exerted through the long form of the receptor.

The predominant form of prolactin has two receptor binding sites and complexes first with one receptor molecule to form a dimer, and then transiently with a second receptor allowing the two receptors and their auxiliary signalling molecules to interact [Gertler et al., 1996]. Signal transduction only proceeds when the receptor-hormone-receptor trimeric complex is formed (FIG. 10).

The in vivo regulation of prolactin receptors is complex and varies between tissues. Changing steady state levels are dependent on the relative rates of synthesis, internalisation and recycling of receptors [Barash et al., 1983; Barash et al., 1986; Posner et al., 1975].

Cellular distribution and abundance of prolactin receptor mRNA is similar to the distribution and abundance of prolactin receptor protein across a range of fetal and adult tissues [Freemark et al., 1993; Maaskant et al., 1996; Royster et al., 1993]. In skin for example, epithelial cells in resting hair follicles show higher immunoreactivity [Choy et al., 1997] and in situ hybridisation signal [Nixon et al., 2002] than in growing follicles. These histochemical studies support the proposition that prolactin receptor is largely transcriptionally regulated, even though the translated products can vary in size from 30 to 95 KDa Maaskant et al., 1996].

Short-term up-regulation by prolactin of its own receptors has been reported in some tissues and tissue explants [Barash et al., 1986; Posner et al., 1975; Rui et al., 1986].

Both up- and down regulation have also been observed, dependent on the dose of prolactin [Rui et al., 1986].

Physiological Effects of Prolactin

Lactation

The increase in prolactin at the time of parturition stimulates the final phases of lactatogenesis and is essential to normal milk production in the subsequent lactation [Ostrom, 1990]. Failure or a delay in onset of lactation occurs when the periparturient prolactin surge is abolished using bromocriptine (an inhibitor of prolactin secretion) in ewes [Fulkerson et al., 1975; Peterson et al., 1991; Peterson et al., 1997; Schams et al., 1984], cows [Akers et al., 1981; Peel et al., 1978], and goats [Forsyth and Lee, 1993]. Infusions of exogenous bovine prolactin can prevent bromocriptine-induced reductions in milk yield in cows [Akers et al., 1981].

Milk yields are lower in bromocriptine treated ewes than in control ewes over the first 4 weeks of lactation [Peterson et al., 1997], while milk protein content is increased. In rabbits, milk yields during an established lactation can be reduced for up to 36 h following a single bromocriptine injection [Mena et al., 1982]. Recovery in milk yield in bromocriptine-treated rabbits is accelerated by single injection of 3 mg of prolactin.

Pregnant dairy heifers or ewes exposed to a long day photoperiod prior to parturition have a significantly larger periparturient prolactin surge relative to those in normal or short day photoperiods [Kendall, 1999; Newbold et al., 1991]. In addition to the well-characterised increase in milk yield of a long-day photoperiod during an established lactation [Dahl et al., 1997; Peters et al., 1978] there is evidence that altered photoperiod during the dry period may affect milk yield during the subsequent lactation [Petitclerc et al., 1998].

Colostrum is defined in the dairy industry as the first milks following calving that have more than 1.45 g/L immunoglobulin G1. Normally colostrum ceases being produced within 4-5 days of calving when copious milk production begins. Administration of bromocriptine to cows, to block the surge in prolactin at calving, both delays the onset of milk production following calving [Akers et al., 1981] and concentrates immunoglobulin G1 in the milk out past a week [Johke and Hodate, 1983]. More recent work shows that prolactin directly down-regulates the immunoglobulin G1 receptor, which transports immunoglobulin G1 into colostrum, in mammary tissue [Barrington et al., 1997].

During the bovine lactation there are also variations in milk composition that are dependent on time of year rather than stage of lactation or nutritional status [Auldist et al., 1998] suggesting a photoperiodic mechanism.

Fibre Growth

Many mammals, including sheep, exhibit seasonal pelage growth [Ling, 1970] that is entrained by photoperiod and mediated by prolactin [Lincoln, 1990; Rougeot et al., 1984].

For example, long-woolled sheep breeds have high wool growth rates in summer and low growth rates in winter which is reflected in the associated variation in fibre diameter and length growth rate [Sumner and Revfeim, 1973; Woods and Orwin, 1988].

Increased variability in diameter along the fibre is, in turn, associated with a reduction in fibre tensile strength. The consequences of this inconsistency, in conjunction with the reduced productivity, extend from farm management and profitability to wool processing and marketing.

While the seasonal growth pattern is driven largely by changes in daylength, the fall in winter wool growth coincides with a pregnancy-induced decline in follicle output [Pearson et al., 1999b] and with reduced pasture availability. The reductions in follicle output during winter and during pregnancy can be ameliorated, but not prevented, by nutritional management [Hawker et al., 1984; Masters et al., 1993; Oddy, 1985] suggesting that hormonal mechanisms may be involved.

Growth and Development

Long days increase growth rates in cattle and accelerate the onset of puberty [Peters et al., 1980]. An effect on nutrient partitioning (increased feed efficiency and lean gain) has also been linked with increased levels of prolactin in cattle and sheep [Schanbacher and Crouse, 1980; Tucker et al., 1984]. There is some direct evidence that prolactin may be anabolic in a number of species [Nicoll, 1980]. The infusion of prolactin increases nitrogen retention in sheep held in darkness [Brinklow and Forbes, 1983], while immunising sheep against prolactin suppresses body growth rates [Ohlson et al., 1981].

Reproduction

Effects of daylength and of prolactin on reproduction are well characterised in many species [Curlewis, 1992; Loudon and Brinklow, 1990; Reiter, 1980; Smith et al., 1987; Soares et al., 1991]. In seasonal breeders, the onset of the breeding season and of puberty can be controlled by manipulation of the photoperiodic environment [Hansen, 1985] and of circulating prolactin [Loudon and Brinklow, 1990; Smith et al., 1987]. In species with an obligate seasonal embryonic diapause (e.g. mink), the seasonal increase in prolactin can have a luteotrophic or luteostatic actions [Curlewis, 1992].

Prolactin also influences the reproductive axis in males. For example, suppression of prolactin in rams with bromocriptine in summer decreases steroidogenic and spermatogenic activity in the testes [Regisford and Katz, 1993; Regisford and Katz, 1994] and causes regression of accessory sex glands [Barenton and Pelletier, 1980].

All references, including any patents or patent applications cited in this specification are hereby incorporated by reference. No admission is made that any reference constitutes prior art. The discussion of the references states what their authors assert, and the inventors reserve the right to challenge the accuracy and pertinency of the cited documents. It will be clearly understood that, although a number of prior art publications are referred to herein, this reference does not constitute an admission that any of these documents form part of the common general knowledge in the art, in New Zealand or in any other country.

It is an object of the present invention to address the foregoing problems or at least to provide the public with a useful choice.

Further aspects and advantages of the present invention will become apparent from the ensuing description which is given by way of example only.

### DISCLOSURE OF INVENTION

According to one aspect of the present invention there is provided a method for affecting a physiological response of an animal to circulating level of prolactin and/or prolactin mimetics, characterised by the step of modulating prolactin receptors.

The term ‘physiological response’ should be taken to mean any physiological response of an animal that arises directly or indirectly as a result of modulating prolactin receptors. This can include physiological processes such as lactation, hair and wool growth, muscle development and/or fertility. It should be appreciated that these are given by way of example only and should not be viewed as limiting in any way, for prolactin is thought to be responsible for as many as 300 different effects on central and peripheral tissues.

The term ‘circulating level’ should be taken to mean the concentration of prolactin circulating in the blood of an animal.

The term ‘prolactin mimetic’ should be taken to mean a molecule which because of its structural properties is capable of mimicking the biological function of prolactin, such as causing prolactin receptor signalling or altering the sensitivity or number of prolactin receptors.

The term ‘prolactin receptor(s)’ refers to any type of receptor to which prolactin is known to bind.

The term ‘modulation’ should be taken to mean the artificial interference on prolactin receptors by a number of means. This may include treatments which either alter the receptor number and/or alter the sensitivity of the receptors to prolactin.

For example, modulation may have a number of effects on prolactin receptors, such as changes in the regulation of prolactin receptor transcription, mRNA stability and translation, or receptor sensitivity. However, this should not be seen as limiting and it should be appreciated that receptor modulation could include a range of other effects.

The modulation of prolactin receptors may be brought about by a number of means. Preferably, the modulation of receptor numbers is brought about by a sustained increase in the initial circulating level of prolactin and/or prolactin mimetics, followed by the decrease back to normal or low levels.

The modulation of prolactin receptors is thought to prime prolactin-dependent physiological processes to a second increase in prolactin levels. This second increase in prolactin may be brought about naturally, for example by an increase in the photoperiod to which an animal is exposed, or may be artificially induced in an animal.

Prolactin is preferably administered by any method which will induce a sustained increase in circulating prolactin, such as by intravenous infusion, by a slow release bolus or implant. However, this should not be seen as limiting and a number of methods known in the art could be used.

The administration may be carried out to effect the initial modulation of the receptors and/or for the later introduction of a second event of elevated prolactin that the modulated receptors respond to.

The inventors have surprisingly found that in order to effect the modulation of physiological processes, the elevated circulating prolactin levels must be sustained, prior to the reduction to normal or low levels after infusion. Merely providing daily injections of exogenous prolactin were not sufficient to alter the response of an animal.

After the temporary increase, the circulating levels of prolactin are preferably reduced to normal or low circulating levels. This reduction may be brought about by either decreasing the photoperiod to which the animal is exposed and/or reducing or terminating the exogenous administration of prolactin or prolactin mimetics. The inventors have found that this profile upregulates the expression of the prolactin receptor gene over a sustained period of time.

In one preferred embodiment of the present invention, the photoperiod, i.e. duration of daylight to which animals are exposed, has been found to have an effect on circulating prolactin levels and thus also on prolactin receptors themselves.

Normal (ND) photoperiod may be defined as the seasonally varying natural daylength. Long day (LD) photoperiod may be defined as 16 h light and 8 h dark; and short day (SD) photoperiod may be defined as 8 h light and 16 h dark.

In preferred embodiments, the photoperiod may be altered in a controlled environment by any of the methods well known in the art.

In other preferred embodiments of the present invention the administration of exogenous prolactin may be given to animals to mimic the effect of increased photoperiod (LD) on the circulating levels of prolactin.

Exogenous purified prolactin from any commercially available source, for example a recombinant product or from a protein extract derived from sheep pituitary glands, is preferably given over an extended period of time.

For example, to alter hair and wool growth in sheep, the inventors have found that the optimum length of sustained prolactin increase is from 3-18 days, more preferably 3-15 days and most preferably 9 days. While it is expected that the effect on hair and wool growth may still occur when the increase in circulating prolactin is sustained for more than 18 days, it is unlikely that such lengthy treatments would be particularly cost effective.

In this example, the concentration of circulating prolactin is preferably first increased by 5 ng/mL-800 ng/mL above normal levels and then returned to normal levels.

Most preferably, the concentration is increased by 5 ng/mL to 200 ng/mL above normal levels.

When endogenous circulating levels of prolactin are low (<50 ng/ml), small increases in prolactin of less than 5 ng/ml may also be effective in inducing changes in prolactin receptor abundance or sensitivity.

It should be appreciated that this is given by way of example only and the lengths of prolactin increase and the concentration thereof should not be seen as a limitation on the present invention in any way. Other species and other tissues are expected to require different optimal conditions.

In another preferred embodiment of the present invention the increase in the circulating level of prolactin may be brought about by the incorporation into the animal's genome of an inducible recombinant nucleotide sequence encoding biologically active prolactin or a recombinant nucleotide sequence encoding a molecule which enhances endogenous prolactin activity. By over expressing prolactin, the timing and level of expression of specific genes may be altered in transgenic animals.

For example, the prolactin gene sequence could be inserted into an inducible gene cassette under the control of a suitable mammary-specific promoter such as a milk protein, a promoter that expresses in all cell types (constitutive expression), or the prolactin promoter and/or a suitable enhancer sequence to drive transcription thereof.

This cassette would also preferably contain 3′ flanking DNA that could stabilise the mRNA and may contain downstream regulatory sequences.

This DNA cassette could be introduced into the genome of an animal by microinjection of the DNA into pronuclei of eggs (described by L'Huillier et al., 1996) which are subsequently transferred back to recipient animals and allowed to develop to term. This technique for the production of transgenic animals is described by Hogan et al. (1994).

Another way to produce transgenic animals involves transfection of cells in culture that are derived from an embryo, or foetal or adult tissues followed by nuclear transfer and embryo transfer to recipient animals. Alternatively, the gene cassette may be bound to mammalian sperm and delivered to the egg via in vitro or in vivo fertilisation to produce a non-human transgenic animal.

Manipulation of the developmental regulation or the level of expression of prolactin may be used to alter the characteristics of the physiological responses of an animal, or alter the rate whereby these occur.

Alternatively, the gene cassette may comprise a DNA sequence encoding a molecule which enhances endogenous prolactin activity or alters the secretion of substances affecting prolactin release or plasma half-life such as prolactin binding proteins, oestrogen, GnRH associated prolactin inhibiting factor, pit-1, hypothalamic dopamine, serotonin and gamma aminobutyric acid or any other suitable molecule as would be known to a person skilled in the art [Freeman et al., 2000].

According to another aspect of the present invention there is provided a method of modulating prolactin receptors by artificially increasing the circulating level of prolactin to a concentration and for a period of time as required, followed by the reduction in the circulating level of prolactin to basal or lower levels.

Preferably, the circulating levels of prolactin are reduced, after the temporary sustained increase, to normal or low circulating levels. This reduction may be brought about by either decreasing the photoperiod to which the animal is exposed, and/or reducing or terminating the exogenous administration of prolactin or prolactin mimetics. The inventors have surprisingly discovered that the reduction in circulating prolactin back to basal or normal levels is important for the modulation of wool growth.

In animals which have an inducible recombinant nucleotide sequence incorporated into their genome which increases prolactin either directly or indirectly, the circulating levels of prolactin may be reduced by the administration of an inhibitor of prolactin synthesis or by cessation of prolactin induction.

In transgenic animals, the induced increase in circulating levels of prolactin may be reduced after a desired period by switching off the inducible gene cassette, and the effects on the physiological processes of an animal measured by known methods.

In another preferred embodiment of the present invention, the modulation of prolactin receptors may be achieved by the administration of antibodies capable of affecting the response of the prolactin receptors to circulating prolactin levels. These antibodies may have stimulatory or inhibitory effects on prolactin receptors, act as prolactin mimetics, or may bind to circulating prolactin/prolactin mimetic molecules to keep these in circulation for longer, prolonging the physiological response.

Immunological manipulations of this type of hormone/receptor system can be applied to animal production (Aston et al., 1991; Pell & Aston, 1995). Inhibitory and stimulatory antibodies to rabbit prolactin receptor have been described (Djiane et al., 1985) as have antibodies that enhance the activity of growth hormone (Holder et al., 1985; Beatie & Holder, 1994) and insulin-like growth factor-I (Hill & Pell, 1998). Again the timing, duration and effective increase and decrease in prolactin-like activity are as described above.

The modulation of prolactin receptors may be induced at any time throughout the year to affect physiological processes of an animal, although it may preferably be given at times when natural circulating prolactin levels are low such as during the winter. It will be understood by a person of skill in the art that the prolactin profile of a species of interest would be of use in deciding the optimum times in which to carry out the method of the present invention.

Knowledge of the prolactin profiles of an animal of interest would be useful in carrying out the method of the invention at the preferred timing, i.e. when natural circulating levels of prolactin are not changing rapidly and it is within the capacity of a person skilled in the art to obtain such a prolactin profile.

However, the inventors have shown that the method of the invention will work even when the natural circulating level of prolactin is high, for example during parturition, so that timing of the modulation of prolactin receptors does not appear to be restrictive. The method of the invention is expected to work throughout the year and not be dependent on the seasonal or pregnancy-induced changes in the prolactin profile of an animal, though these changes can be used to affect the physiological response of an animal after the initial modulation of prolactin receptors.

The present invention also provides an animal treated by the method of the invention including transgenic animals and their off-spring.

According to a further aspect, the invention provides animal products produced by an animal treated by the method of the invention.

The inventors have also devised a model to predict the effect of a particular treatment as a guideline for developing the best method for treatment or of experimental design.

Using this model, it is possible to determine parameters for a particular species or tissue and to design the optimal timing, plasma profile and dosage of a temporary but sustained prolactin (or a mimetic) treatment or immunological manipulation to alter short and long-term physiological responses to these.

The model can also be used to predict the appropriate timing, plasma profile and dosages of single or serial, temporary sustained prolactin (or a mimetic) treatment(s) or immunological manipulation so as to modulate prolactin receptors to optimally enhance responses to prolactin or to prolactin mimetics.

In other embodiments, the model may be used to design the appropriate timing, plasma profile and dosages of single or serial, temporary sustained prolactin (or a mimetic) treatment(s) or immunological manipulation so as to modulate prolactin receptors to optimally inhibit responses to prolactin or to prolactin mimetics.

It should be appreciated that other models may be developed which can predict changes in receptor numbers, receptor sensitivity and/or other receptor parameters.

Specifically, the inventors have devised a mathematical model of prolactin-prolactin receptor interaction, developed from knowledge of receptor dynamics in general and prolactin receptor biochemistry in particular. By using experimental data showing long-term prolactin receptor gene transcription induced by circulating prolactin, a number of experimentally observed phenomena were predicted.

Firstly, the necessity for a rise, a sustained elevated concentration and then a decline in circulating prolactin concentration for optimal short-term biological effects and for longer term stimulatory effects on prolactin receptor numbers (and therefore enhanced biological responsiveness to prolactin or prolactin mimetics) were predicted. In addition, the ineffectiveness of prolactin injections versus the enhanced responses to sustained prolactin infusions on prolactin receptor numbers was also predicted.

Another experimentally observed effect predicted by the inventors' model was that a long term depression of prolactin receptor numbers and therefore diminished biological responsiveness to prolactin or prolactin mimetics resulted in response to very high or prolonged prolactin (or prolactin mimetic) treatment.

### DESCRIPTION OF THE DRAWINGS

The invention will be further described by reference to the figures of the accompanying drawings (pages 1/19-19/19) in which:

FIG. 1 shows the experimental design for the six trials using sheep disclosed herein. The length of the bars represents the duration of each treatment as shown in the legend; SD means short days; ND means natural days; LD means long days. NP means non-pregnant (dry) ewes; L means breeding (lambed) ewes; BrB means bromocriptine administered to breeding ewes before parturition; BrA means bromocriptine administered to breeding ewes after parturition; PRL-INF means prolactin administered intravenously and PRL-INJ means prolactin injected subcutaneously;

FIG. 2 shows the effect of photoperiod manipulation on circulating prolactin and prolactin receptor expression for Trial 1. The change from short- to long-day photoperiod (SD:LD) on 15 January (Southern Hemisphere summer) is indicated by an arrow. Top panel: mean plasma prolactin concentrations measured by radioimmunoassay from morning samples: (▪) values for control animals exposed to natural day length, (♦) values for light-treated animals. Bars show the standard error of the mean. Bottom panel: relative abundance of mRNA for long form (●) and short form (▾) of PRLR determined by RNase protection assay from animals sacrificed throughout the experiment. Lines follow averages of duplicate animals.

FIG. 3 shows the midside mean fibre diameter (top panel), midside clean wool growth rate (second panel), plasma prolactin and midside clean wool growth rate for selected groups (third and fourth panels) and the mean clean fleece weight (±standard error of the mean) collected at shearing (bottom panel) for each treatment group of Trial 2. Key: (□) ND non-pregnant; (▪) ND-lambed; (▴) ND-BrB; (▾) ND-BrA; (◯) LD non-pregnant; and (●) LD-lambed. Bottom panel: □ ND non-pregnant; ▪ ND-lambed; [symbol] ND-BrB; [symbol] ND-BrA; [symbol] LD non-pregnant; and [symbol] LD-lambed ewes. The vertical error bar in the first and second panels shows the pooled SED of the means.

FIG. 4 shows the mean plasma prolactin concentration of the treatment groups of Trial 3 for (●) LD-lambed, (▪) ND-lambed and (□) ND non-pregnant ewes (top panel); (♦) PRL-INJ ewes (middle panel) and (▴) PRL-INF ewes (bottom panel). Prolactin administration was for 18 days indicated by the hatched bar.

FIG. 5 shows for each of the treatment groups of Trial 3, the mean fibre diameter (top panel) and the mean clean wool growth rates (middle panel) (Key: (●) LD-lambed; (♦) PRL-INJ; (▴) PRL-INF; (▪) ND-lambed and (□) ND non-pregnant ewes); and the mean clean fleece weight (±standard error of the mean) collected at shearing (bottom panel) (Key: □ ND non-pregnant; ▪ ND-lambed; [symbol] PRL-INF; [symbol] PRL-INJ and [symbol] LD-lambed ewes). Prolactin administration was for 18 days indicated by the hatched bar.

FIG. 6 shows for each of the treatment groups of Trial 3, the mean relative skin PRLR (long form) mRNA expression measured by real-time PCR. Prolactin administration was for 18 days indicated by the hatched bar. Key: (◯) non-pregnant ewes, (♦) pregnant ewes, (▴) infused pregnant ewes, (●) injected pregnant ewes and (▪) are LD pregnant ewes. Vertical bars show SED between means for each sampling date.

FIG. 7 shows the mean plasma prolactin concentration of the treatment groups of Trial 4 for (□) ND non-pregnant ewes (top panel); (▪) 3-day PRL infusion (second panel) (♦) 9-day PRL infusion (third panel) and (▾) 18-day PRL infusion ewes (bottom panel). Prolactin administration was for the periods indicated by the hatched bars. P represents the mean date of parturition; W represents the date of weaning;

FIG. 8 shows for each of the treatment groups of Trial 4, the mean fibre diameter (top panel) and the mean patch clean wool growth rates (middle panel) (Key: (▪) ND-lambed—estimated from non-pregnant ewes; (♦) 3-day PRL infusion; (●) 9-day PRL infusion; (▾) 18-day PRL infusion); and the mean total clean patch weight (±standard error of the mean) collected over the trial (bottom panel) (Key: ▪ ND-lambed—estimated from non-pregnant ewes; [symbol] 3-day PRL infusion; [symbol] 9-day PRL infusion and [symbol] 18-day PRL infusion ewes). Prolactin administration was for the periods indicated by the hatched bars.

FIG. 9 shows for each of the treatment groups of Trial 4, the mean relative skin PRLR (long form) mRNA expression measured by real-time PCR. Prolactin administration was for 3, 9 and 18 days indicated by the hatched bars. Key: (▪) pregnant ewes, (Δ) 3-day infused pregnant ewes, (δ) 9-day infused pregnant ewes and (◯) 18-day infused pregnant ewes. Vertical bars show SED between means for each sampling date.

FIG. 10 shows the mean plasma prolactin concentrations (top panel) and the relative log prolactin receptor mRNA concentrations (bottom panel) of the treatment groups of Trial 5: (▪) ND ewes; (◯) SD saline infused; and (♦) SD prolactin infused ewes. The vertical error bars in the second panel show SED between means for each sampling date. Prolactin administration was for the periods indicated by the hatched bars.

FIG. 11 shows the mean plasma prolactin concentrations (top panel) and the relative log prolactin receptor mRNA concentrations (bottom panel) of the treatment groups in Trial 6. Key: (Δ) SD Romney ewes; (♦) SD prolactin infused Romney ewes; (◯) SD Wiltshire ewes; and (▪) SD prolactin infused Wiltshire ewes. The vertical error bars in the second panel show SED between means for each sampling date. Prolactin administration was for the periods indicated by the hatched bars.

FIG. 12 shows the relative log prolactin receptor mRNA concentrations in the liver, mammary gland and skin of rabbits in Trial 7 before and after a 7-day infusion of ovine prolactin.

FIG. 13 shows for each of the treatment groups of Trial 8 the mean plasma prolactin concentrations (top panel) and the relative log prolactin receptor mRNA concentrations (bottom panel) (±standard error of the mean). Key: (◯) saline and bromocriptine control group; 3-day prolactin infusion group (▴); and 7-day prolactin infusion group (▪).

FIG. 14 shows the schematic structure of a mathematical model of prolactin receptor regulation by prolactin. The four output variables are the concentration of plasma prolactin, and the numbers of unbound, and bound receptors as dimeric and trimeric complexes (shown in boxes). The number of bound receptors increases due to association of prolactin with unbound receptors and decreases because of degradation (D) and dissociation back to the unbound state. The number of unbound receptors decreases because of binding and degradation (D), and increases due to synthesis (S) and dissociation processes. The concentration of plasma prolactin decreases mostly because clearance (D) whilst secretion and artificial prolactin input cause an increase. These statements are expressed as a set of linked differential equations (see text).

FIG. 15 shows a mathematical simulation of the concentrations of unbound and total receptors (top panel, dashed and bold lines respectively), signalling trimer complex (middle panel), and concentration of plasma prolactin (bottom panel) with a nine day infusion of prolactin starting at day 5.

Initial conditions are determined by equilibrium at assumed constant values and parameter settings. Because no prolactin is being infused over the initial ten days, the plasma prolactin concentration and the number of bound and unbound receptors do not change. The number of unbound receptors drops as the prolactin is infused, with a corresponding rise in bound receptor number. Once the infusion is switched off, the number of bound receptors declines back to the initial equilibrium, and the number of unbound receptors reaches its highest level, then slowly relaxes to the equilibrium level.

FIG. 16 shows a mathematical simulation of the concentrations of unbound and total receptors (top panel, dashed and bold lines respectively), signalling trimer complex B₂ (middle panel), and concentration of plasma prolactin P (bottom panel) with a nine day infusion of prolactin. Conditions are as for FIG. 15, except that the prolactin pulse occurs after 17 days. The changes in level of receptors are independent of the timing of the pulse of prolactin.

FIG. 17 shows a mathematical simulation of the numbers of unbound and total receptors (top panel, dashed and bold lines respectively), bound receptors in signalling trimer complex B2 (middle panel), and concentration of plasma prolactin P (bottom panel) with two successive nine-day infusions of prolactin. The first infusion alters the conditions for the second infusion resulting in higher levels of bound receptors after the second infusion.

FIG. 18 shows a mathematical simulation of the numbers of unbound and total receptors (top panel, dashed and bold lines respectively) and bound receptors in signalling trimer complex B2 (middle panel) in response to a series of prolactin injections followed by a nine-day infusion of prolactin. Prolactin concentration is shown in bottom panel. Injections cause elevations of prolactin which are greater than that caused by infusion, but with much smaller duration, and result in a comparatively smaller response, showing the system is not sensitive to short-term increases in prolactin.

FIG. 19 shows that the peak trimer concentration, and therefore the potential biological response, resulting from a 9-day infusion of prolactin varies in a non-linear fashion with the elevation of prolactin over its basal level. There is an optimal infusion level for any infusion duration, indicated by the peak, after which signalling level starts to decrease.

### DETAILED DESCRIPTION OF THE INVETION

As defined above, the present invention is directed to affecting a physiological response of an animal to circulating levels of prolactin and/or prolactin mimetics by modulating prolactin receptors.

The invention is based upon the inventors' investigation into the profiling of prolactin, the effect of prolactin upon prolactin receptors and the development of a model to predict these effects and allow better methods of treatment and experimental design.

Non-limiting examples of the invention will now be provided.

Protocol

Eight trials are described to demonstrate the principles of the invention by describing the effects of a temporary sustained prolactin surge on short and long term prolactin receptor gene expression, and by experimentally manipulating plasma prolactin profiles in sheep causing changes in annual wool growth patterns.

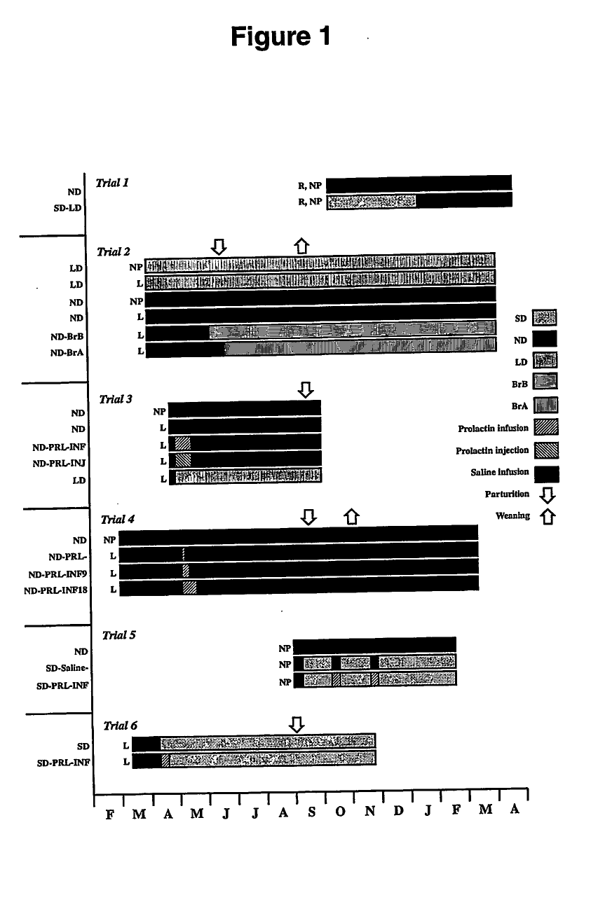

Two breeds of sheep were utilised and showed differing effects related to their wool growth characteristics. The New Zealand Romney grows long relatively course wool continuously, but with marked seasonal variation in growth rate. The research conducted by the inventors showed that prolactin modulates the seasonal production pattern of this breed. By contrast, the New Zealand Wiltshire is a meat breed with slow, discontinuous fleece growth. In these experiments, prolactin treatments altered the timing of hair cycles and fleece shedding. Romney sheep were used in trials 2 to 6. Wiltshire sheep were used in trials 1 and 6. The designs for these trials are shown in FIG. 1.

The research was carried out in the Southern Hemisphere where summer, autumn, winter and spring correspond to the calendar months December-February, March-May, June-August and September-November respectively. The principal objectives were to:
to determine the effects of a temporary sustained prolactin surge (induced by long day photoperiod) on the regulation of prolactin receptor gene transcription (Trial 1);
to determine the effect of differing profiles of endogenous prolactin on wool growth (Trial 2);
to determine the effect of a sustained, temporary increase in the plasma concentration of prolactin, induced by infusion of exogenous prolactin, on subsequent wool growth patterns (Trials 3 and 4);
to determine the effects of two different modes of administration (injection versus infusion) on subsequent wool growth (Trial 3);
to show that both stimulatory and inhibitory wool growth effects can be achieved depending on the dose and mode of administration (Trials 3 and 4).
to show that two successive periods of exogenous prolactin administration to non-pregnant ewes can perturb prolactin receptor gene expression on each occasion (Trial 5).
to show that prolactin effects on prolactin receptor gene expression depend on genotype (Trial 6).
to show that prolactin administration can perturb prolactin receptor gene expression in other species apart from sheep and in other tissues apart from skin (Trials 7 and 8).

For Trials 2-6 and 8 the data relating to the wool growth measurements, plasma prolactin concentrations and prolactin receptor gene expression were subjected to an analysis of variance at each sampling time to test the effects of treatment. Plasma prolactin and prolactin receptor gene expression values were log transformed before analysis to allow assumption of homogeneous variance in all experimental groups.

For the wool growth data, initial values were used as covariates for analysis.

Trial 1

Long-Term Regulation of Prolactin Receptor Gene Transcription by Prolactin

Wool follicle cycles were synchronised in New Zealand Wiltshire sheep using an artificial photoperiod regime to manipulate circulating prolactin, as previously described [Parry et al., 1996].

Twenty-nine mature sheep (18 rams and 11 ewes) were maintained indoors on a constant diet of sheep pellets and hay for six months from 11 October (Southern Hemisphere spring). The animals were allocated to one of two groups. Group 1 (n=9; 4 rams and 5 ewes) was exposed to normal daylight via windows. Group 2 (n=20; 14 rams and 6 ewes) were exposed to a constant short day length (L8:D16) for 13 weeks and then, from 15 January (day 0), to long day length (L16:D8) until 23 April (day 98).

Such an artificial lighting regime has been shown to abolish the normal spring rise in pituitary prolactin secretion, then, with the photoperiod transition in mid summer and release of prolactin suppression, to synchronously induce follicle regression and interrupt wool growth [Nixon et al., 1997; Pearson et al., 1993].

Blood samples (5 ml) were collected from all animals by jugular venipuncture at 2 to 10 day intervals from 22 October (85 days prior to the change of photoperiod) until 22 April (day 97 after change of photoperiod). Prior to the change in photoperiod at day 0, blood samples were taken in the morning between 08:00 and 09:30. After day 0, blood was also collected in the evening between 20:00 and 21:30. Plasma was separated by centrifugation within 2 hours of blood collection.

Two control sheep from Group 1 were sacrificed on each of days 0, 28, and 98. Photoperiod treated sheep from Group 2 were killed over the course of the induced wool growth cycle; two on each of days 0, 7, 14, 21, 28, 47 and 98. Samples of skin from the mid-sides of these animals were frozen in liquid nitrogen and stored at −85 C. or fixed in phosphate buffered 10% formalin. Fixed skin was processed to paraffin wax and 7 μm transverse sections cut and stained by the Sacpic method for determination of follicle activity [Nixon, 1993]. Plasma prolactin concentrations were measured in duplicate by radioimmunoassay as previously described [Nixon et al., 1993].

Ribonuclease Protection Assays

Total RNA was isolated from approximately Ig of each frozen skin sample collected from Groups 1 and 2 by grinding to powder under liquid nitrogen in a freezer mill (SPEX 7700, Glen Creston Ltd, Middlesex, UK), and extracting with “TRIzol” reagent (Gibco BRL, Rockville, Md.) according to the manufacturer's instructions. RNA concentration was measured by spectrophotometry at 260 nm and integrity verified on an agarose/formaldehyde gel.

Antisense riboprobes for ovine prolactin receptor [Anthony et al., 1995] and glyceraldehyde-3-phosphate dehydrogenase (GAPDH) (Genbank accession no. AF022183) were used in ribonuclease protection assays. The prolactin receptor cDNA sequence spanned an alternatively spliced region in the proximal cytoplasmic domain and was therefore able to distinguish RNA variants encoding long and short isoforms of prolactin receptor indicated by protected fragments of 441 bp and 549 bp respectively [Choy et al., 1997]. The GAPDH cDNA, encoding 424 bp of the 5′ region, was generated by RT-PCR of sheep skin and cloned into pGemT vector (Promega, Madison, Wis.).

Both riboprobes were labelled with a-33P-uridine 5′-triphosphate (Amersham International, Buckinghamshire) by in vitro transcription from linearised plasmids using the Riboprobe Core System (Promega, Madison, Wis.).

RNase protection assays of both prolactin receptor and GAPDH were carried out in duplicate using the Ambion RPAII Kit (Ambion, Austin, Tex.) following the manufacturer's instructions. Forty micrograms of total RNA was hybridised with both riboprobes at 45° C. overnight. Unhybridised RNA was removed by RNase digestion followed by inactivation of RNase and precipitation of protected fragments. These fragments were separated by electrophoresis on a 5% polyacrylamide/8M urea gel. After drying, gels were exposed in intensifying screens to Kodak XAR film (Eastman Kodak, Rochester, N.Y.). Optical density of protected fragments was measured using Molecular Analyst Software (BioRad Laboratories, Hercules, Calif.) and prolactin receptor bands were standardised against GAPDH measurements.

Results

Photoperiod Manipulation Altered Prolactin Secretion and Induced a Wool Growth Cycle

In control animals exposed to normal changes in day length (Group 1), the maximum mean plasma prolactin concentration was observed on 6 November (mean±SEM: 148±30 ng/ml) (data not shown). Prolactin levels then gradually declined over the experimental period, although some fluctuations occurred in association with animal management events and unusually high daytime summer temperatures. By comparison, circulating prolactin was suppressed (P<0.001) in animals maintained in short days during spring (Group 2). Mean values at 15 days before the change in photoperiod were 6±2 ng/ml and 7±1 ng/ml in Group 2.

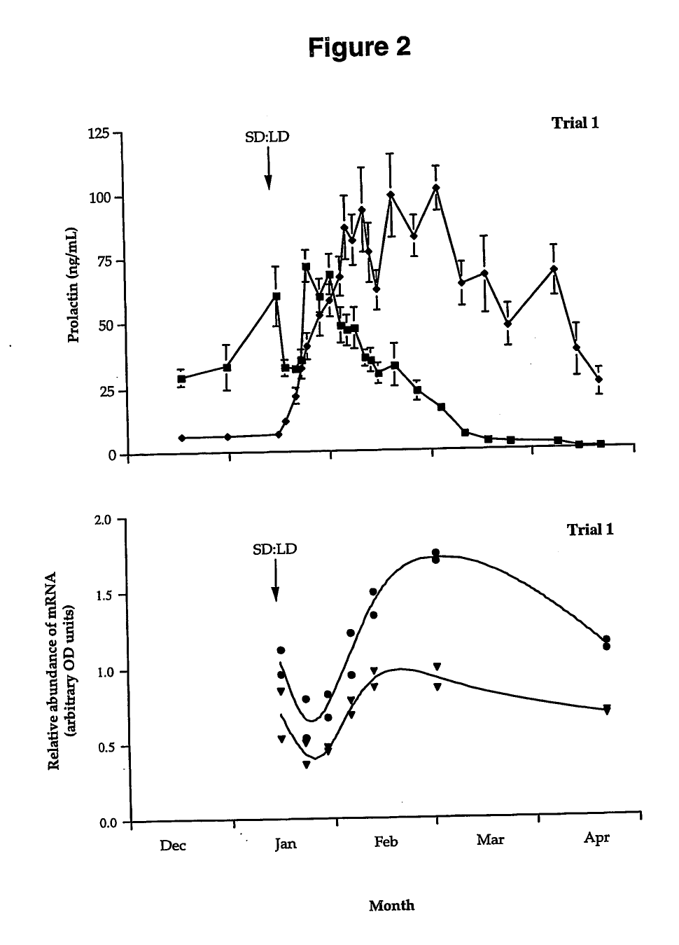

Following transition into long days (day 0), prolactin levels increased rapidly (FIG. 2). By 23 days after the change in photoperiod, concentrations were 81±12 ng/ml in Group 2. Peak evening prolactin levels ranging from 134 ng/ml to 260 ng/ml were observed in individual animals between 23 days and 70 days. Thereafter, plasma prolactin concentration declined in all treated animals (FIG. 2).

Histological assessment of hair follicle growth status showed that virtually all follicles sampled from both treated and control animals at day 0 were in anagen (growth phase) (data not shown). Follicles remained growing in control animals (Group 1) whereas follicles in light treated animals (Group 2) passed through a hair cycle.

By day 28, the majority of follicles sampled from animals undergoing light treatment had entered telogen (resting phase) and in two individuals the percentage of growing follicles had reached a nadir of 2%. The wool follicles progressively reactivated such that by day 98 almost all had resumed growing.

Level of Prolactin Receptor Expression During a Prolactin Induced Wool Follicle Cycle

The abundance of prolactin receptor mRNA relative to GAPDH mRNA in the skin of Group 2 sheep varied following the photoperiod transition and consequent rise and fall in plasma prolactin (FIG. 2). The sampling points covered the major divisions of the hair follicle cycle. The initial response, occurring between day 0 and day 7, was an apparent decline in prolactin receptor mRNA. This corresponded to a small increase in plasma prolactin (P<0.01) of less than 10 ng/ml at five days after the photoperiod transition. No changes in skin or follicle morphology were yet visible, but this time immediately preceded catagen.

From day 7 to day 47, prolactin receptor was up-regulated (long form: P<0.01) (FIG. 2). Over this period, there was a rapid and continuous increase in plasma prolactin concentration and regression of follicles to the telogen phase resulting in the shut down of fibre growth. The transition through catagen saw the most rapid changes in prolactin receptor mRNA levels. Prolactin receptor mRNA was most abundant at day 47 by which time hormone levels were about to fall.

By day 98, circulating prolactin had dropped and prolactin receptor mRNA approached the levels observed at the start of the experiment when follicles were similarly in anagen. The relative abundance of prolactin receptor mRNA in the skin of Group 1 (control) animals did not significantly differ from Group 2 animals when follicles were in anagen (day 0 and day 98) (data not shown).

In all RNase protection analyses, bands corresponding to prolactin receptor long form protected fragment emitted more signal than those of short form protected fragments, indicating the greater abundance of long form transcripts. Both isoforms underwent a similar pattern of decrease followed by increase and return to anagen levels over the prolactin-induced cycle (FIG. 2). The ratio of long- to short form mRNA was greater at 47 days when total prolactin receptor expression was at a maximum and the follicles were in proanagen, as compared with samples in anagen (P<0.05).

Trial 2

Effects of Differing Endogenous Prolactin Profiles on Wool Growth

Natural and experimental changes in plasma prolactin concentration and their effects on wool production in winter-lambing Romney ewes were measured.

Fourteen non-pregnant and 29 pregnant ewes were maintained indoors from early April 1995 for 12 months under controlled photoperiod and controlled dietary intake. Two groups (n=8) were held under long day photoperiod (16L:8D; LD non-pregnant and LD-lambed) while 2 others were exposed to natural photoperiod (ND non-pregnant, n=6 and ND-lambed, n=7). Two further groups (n=7) of pregnant ewes housed in natural days were treated with bromocriptine, either from 1 week before parturition (ND-BrB) or 1-3 days after parturition (ND-BrA), to suppress prolactin secretion.

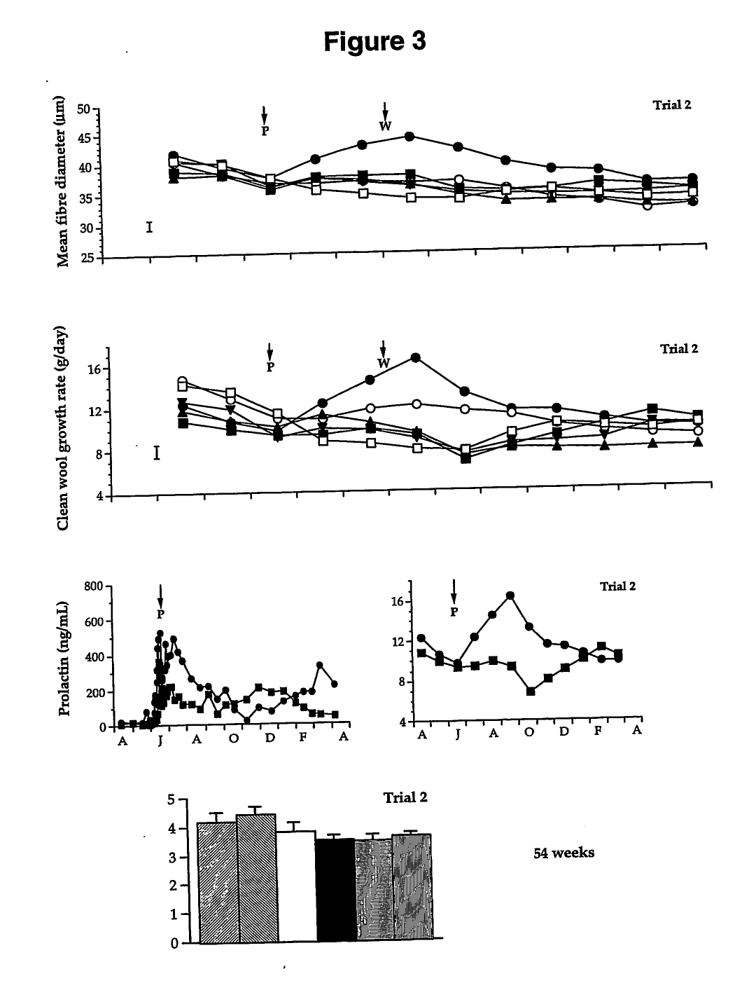

Lambing occurred between 12 and 18 June. Plasma prolactin, mean fibre diameter and wool growth were measured at regular intervals and the results were plotted in FIG. 3.

Results

Photoperiod and treatment with bromocriptine did not affect the birth weight or live weight changes of lambs. In ND non-pregnant ewes, changes in plasma prolactin concentrations were associated with seasonal changes in day length over the duration of the trial. In LD pregnant ewes, continued exposure to LD photoperiod from April caused a significant increase in pre-parturition prolactin concentrations over the winter months compared to ewes held in normal days (24 vs. 11 ng/ml; P<0.02).

Prolactin concentrations were low in the ND-BrB group throughout the trial, and in the ND-BrA group following the prolactin peak associated with parturition (data not shown). Apart from the ND-BrB group, prolactin concentrations in pregnant ewes increased rapidly a few days prior to parturition and subsequently remained elevated. Prolactin concentrations over parturition and lactation were highest in LD-lambed ewes (FIG. 3).

Despite comparable maternal live weights, lambing was associated with a lower clean fleece weight, and a reduced mean fibre diameter, staple length and staple tensile strength compared to non-pregnant groups. LD ewes grew significantly more clean wool than their ND counterparts (P<0.01) as a consequence of an increase in both mean fibre diameter and length growth rate (FIG. 3). The larger prolactin surges associated with parturition and early lactation followed by a decline over mid to late-lactation had a significant stimulatory effect on both wool growth rate (P<0.001) and fibre diameter (P<0.001) in LD-lambed ewes from June to September relative to ND-lambed ewes.

The complete absence of the peripartum prolactin surge was associated with longer-term inhibitory effects on wool growth rate. However, elevated plasma prolactin concentrations during pregnancy, and at parturition and early lactation, followed by a decline in prolactin levels over mid to late-lactation, are linked to higher wool production arising from increases in mean fibre diameter and fibre length growth rate.

Trial 3

Effects of Differing Prolactin Priming Profiles on Long-Term Wool Growth and Prolactin Receptor Gene Expression

The effects of two methods of administration of exogenous prolactin on wool growth were examined.

Forty-three mixed age Romney ewes were maintained indoors in individual pens from April until October, and fed to maintain a constant maternal live weight (independent of conceptus and fleece weights). A control group (ND non-pregnant) was not mated (n=8). Thirty-five other ewes (comprising Groups 2-5) were mated in April. Groups 1-4 were maintained in natural photoperiod while Group 5 (LD-lambed, n=10) was subjected to long days (16L:8D) from 1 week after mating for the duration of the trial (ie 18 days). Group 2 (ND-lambed, n=11) did not receive exogenous prolactin.

An ovine prolactin pituitary extract (0.80 mg/kg/day) was administered by either daily subcutaneous injection to sheep of Group 3 (PRL-INJ, n=10) or by intravenous infusion via a jugular cannula to sheep of Group 4 (PRL-INF, n=2) (from one week after mating for a temporary period of 18 days).

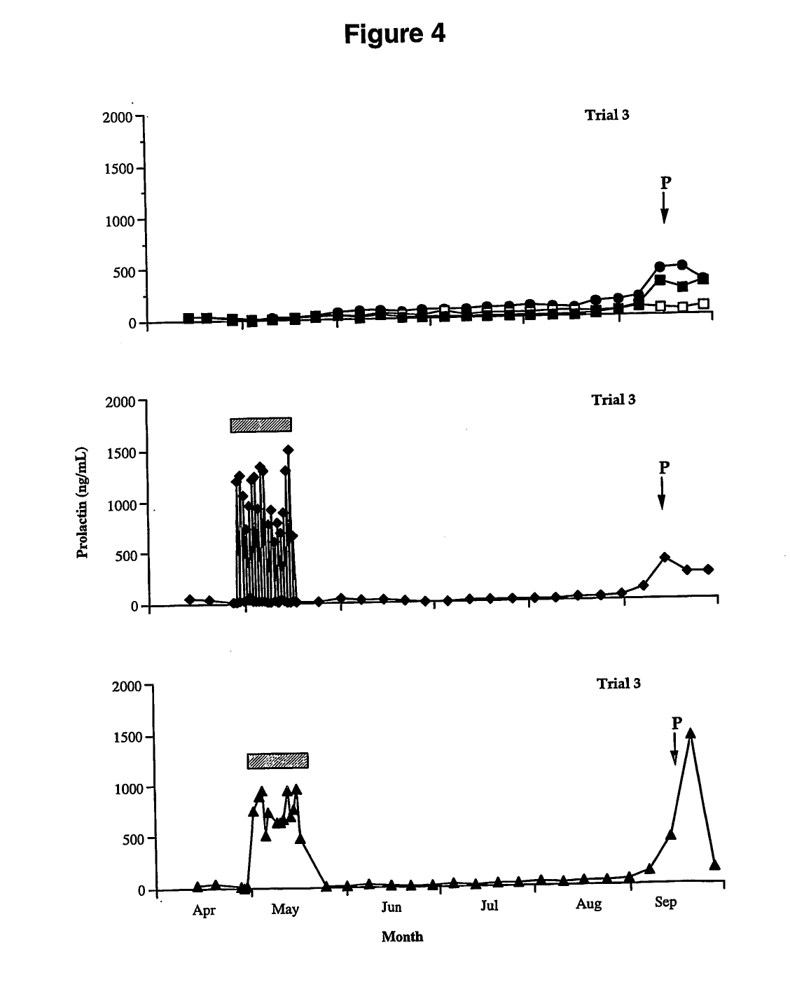

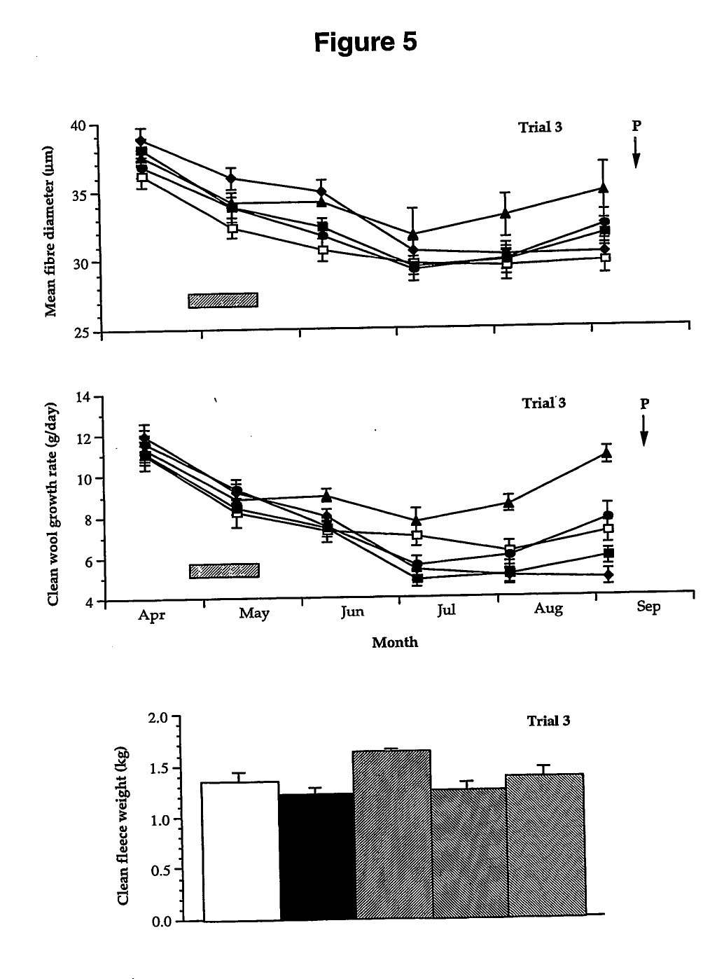

The sheep were shorn on entry to the experiment and again at the conclusion of the experiment in late September and a mid-side wool sample was measured for fibre diameter and yield. Wool samples were clipped from a standardised 100×100 mm mid-side patch on the right side of each ewe at monthly intervals to determine changes in seasonal clean wool production and mean fibre diameter. Wool growth and plasma prolactin concentrations were measured at regular intervals and the results were plotted in FIGS. 4 and 5.

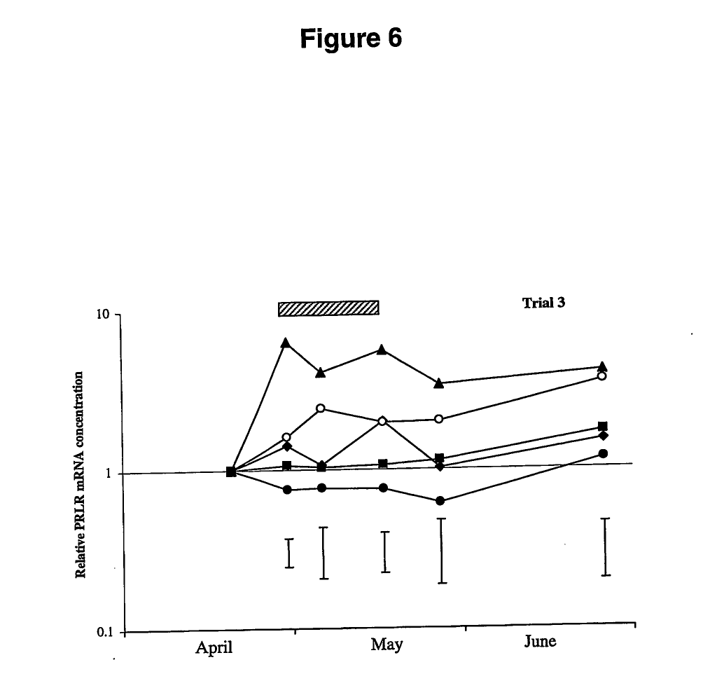

Six midside skin biopsies were collected at intervals between April and June for measurement of prolactin receptor long form gene expression by real-time PCR and the results are plotted in FIG. 6. An assay for the short form of the ovine prolactin receptor was also developed, but results are not presented since only very low levels of expression were detected.

Real-Time PCR Assay for Prolactin Receptor Long Form Gene Expression

The relative expression of prolactin receptor mRNA was measured using Taqman real-time PCR (Applied Biosystems). RNA was extracted from skin samples using TRIzol reagent according to manufactures protocol (Invitrogen). Total RNA was then quantified using RiboGreen reagent (Invitrogen) and all samples were standardized to a concentration of 0.5 μg/μl of total RNA. The reverse transcriptase reaction (Superscript II RT-PCR kit, Invitrogen) was used to generate single stranded cDNA from 0.5 μg of total RNA.

The primers and probes for the long form of the prolactin receptor were designed using the Primer Express program (Applied Biosystems). Primers sequences were as shown in Table 2.

The 18S ribosomal RNA pre-developed assay reagent (Applied Biosystems) was used as an internal control and the reactions were set up according to the manufacturers instructions. Data was analysed using the software provided by Applied Biosystems. Results on relative mRNA quantities were obtained by the standard curve method.

Results

Of the 35 mated ewes, 2 did not produce a lamb and were excluded from the results. Lambing occurred between 9 and 19 September. Prolactin profiles in the control and treated groups are shown in FIG. 4. There was no evidence that prolactin treatment had any adverse effect either on pregnancy maintenance or on the health and growth rate of the lambs (data not shown).

At the commencement of the experiment the clean wool growth rate did not differ between groups, however by September a strong treatment effect was evident (FIG. 5; P<0.001). Clean wool growth rates declined in all groups between April and May, indicative of the winter decline.

An effect of pregnancy was also reflected in the lower clean wool growth rates of mated ewes as compared to the non-pregnant group. Daily subcutaneous injections of prolactin (0.8 mg/kg/day) had no initial effect on the decline in clean wool growth rate compared to the pregnant controls. However, the September clean wool growth was lower (P<0.05) in PRL-INJ ewes than in untreated ND-lambed ewes.

The clean wool growth rate in the LD-lambed ewes did not differ from ND-lambed ewes but showed a tendency to rise more rapidly in August. By September LD-lambed ewes had a significantly higher (P<0.025) clean wool growth rate than their ND-lambed controls. In contrast to other treatment groups the decline in clean wool growth rate in the two PRL-INF ewes was arrested in May and remained high (even above that of non-pregnant control ewes) throughout pregnancy before rising in September to approximately twice that of untreated ND-lambed ewes (P<0.001).

At the commencement of the experiment the mean fibre diameter did not differ between groups, however by September a treatment effect was evident (FIG. 5; P<0.025). Among treated groups, the response pattern of mean fibre diameter was similar to that of the clean wool growth rate. By September, an increase of 2 μm in mean fibre diameter (P=0.05) was observed in the PRL-INF group. The mean fibre diameter appeared to be lower (P<0.10) in the PRL-INJ group relative to ND-lambed controls.

Clean fleece production over the 6-month duration of the trial differed significantly between treatment groups (FIG. 5). Mean clean fleece weight for PRL-INF ewes was greater than that of ND-lambed controls (1.63 vs. 1.23 kg, standard error of the mean±0.13 kg, P<0.01).

Prolactin receptor gene expression in the infused ewes was significantly elevated during and after the 18 day prolactin infusion (P<0.01) compared to the other treatment groups (FIG. 6). The high level of receptor expression in infused ewes with respect to untreated ewes was sustained for two months April to June (P<0.05).

There were no significant differences measured between the remaining treatment groups over the course of the trial.

Trial 4

Effect of Differing Periods of Prolactin Treatment on Long-Term Wool Growth and Prolactin Receptor Gene Expression

Trial 4 was conducted to compare different periods of exogenous prolactin administration on wool growth.

Eighteen 3 year old Romney ewes were maintained indoors in individual pens under natural photoperiod for 12 months from March 2000. Data from a further 6 ewes were excluded on animal health grounds or because they failed to become pregnant. The ewes were fed to maintain a constant maternal live weight (independent of conceptus and fleece weights). A control group (ND non-pregnant) was not mated (n=6). Twelve other ewes (comprising Groups 2-4) were mated on 23 April.

An ovine prolactin pituitary extract (1.0 mg/kg/day) was administered by intravenous infusion via a jugular cannula commencing 11 days after mating (4 May) for temporary periods of either 3 days (Group 2, n=6), 9 days (Group 3, n=3) or 18 days (Group 4, n=3).

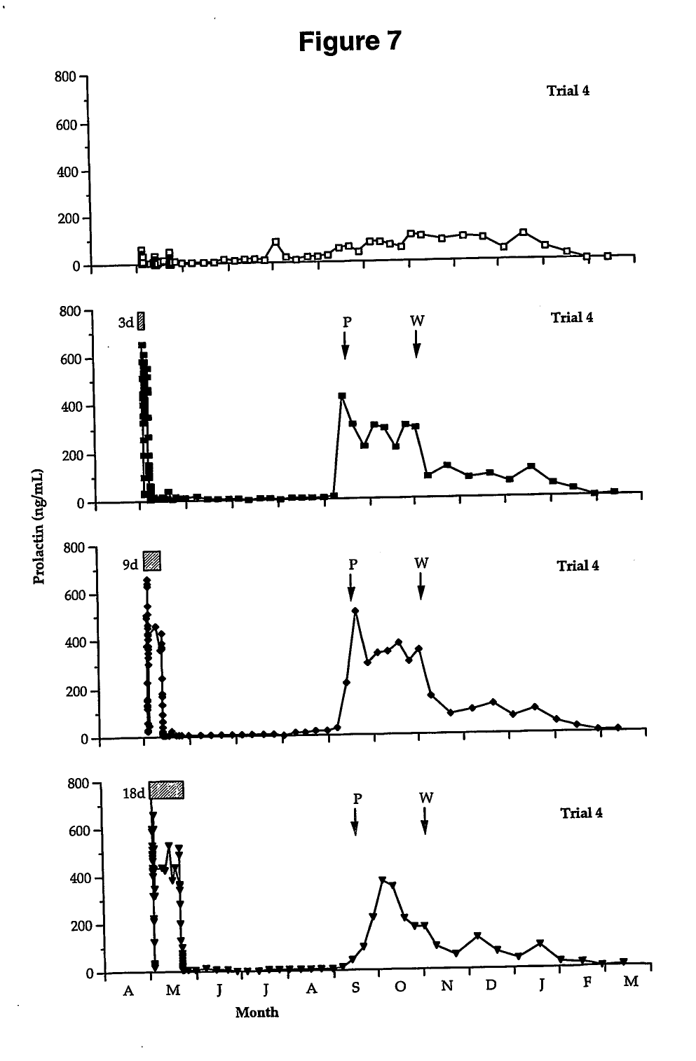

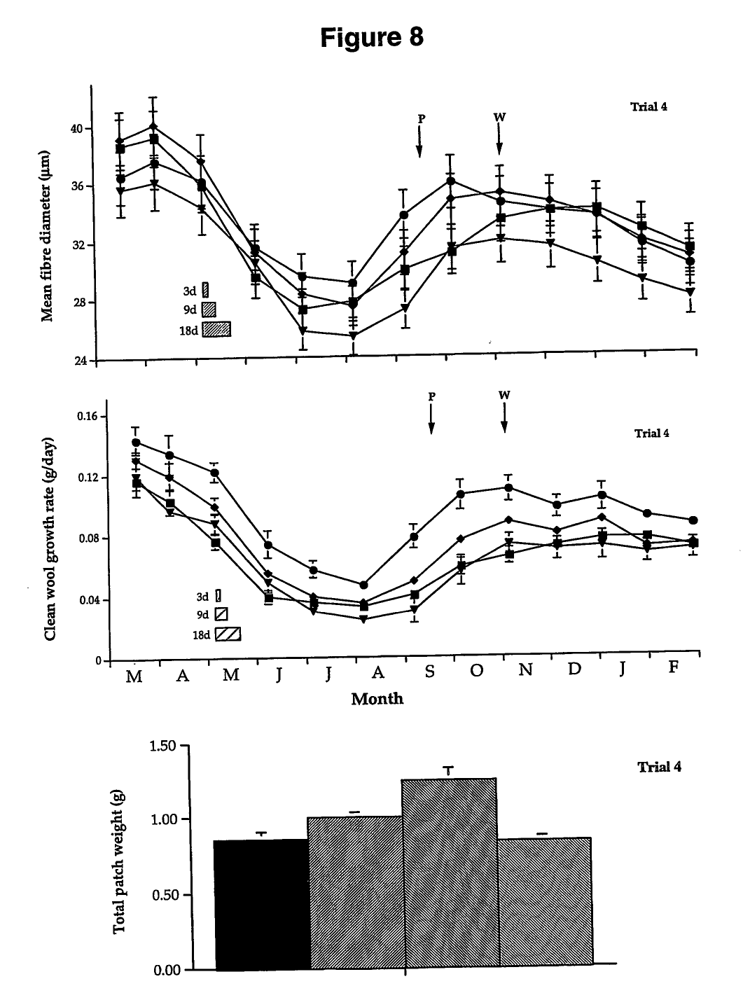

The sheep were shorn on entry to the experiment and again at the conclusion of the experiment in March 2001 and a mid-side wool sample was measured for fibre diameter and yield. Wool samples were clipped from a standardised 100×100 mm mid-side patch on the right side of each ewe at monthly intervals to determine changes in seasonal clean wool production and mean fibre diameter. Wool growth and plasma prolactin concentration were measured at regular intervals and the results were plotted in FIGS. 7 and 8.

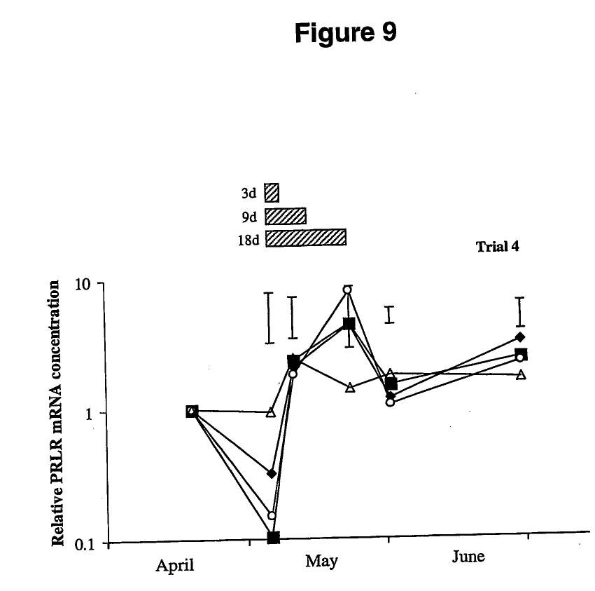

Six midside skin biopsies were collected at intervals between April and June for measurement of prolactin receptor long form gene expression by real-time PCR and the results are plotted in FIG. 9.

Results

Lambing occurred between 14 and 21 September. Clean patch wool growth rates declined in all groups between March and August indicative of the winter decline. Prolactin profiles over the trial in the control and infused groups are shown in FIG. 7.

Prior to the prolactin infusion, clean patch wool growth rate did not differ between groups. However, during May, a short-term treatment effect was evident with higher wool growth in the 9-day infused group compared to either the 3-day (P<0.01) or the 18-day (P<0.02) infused groups (FIG. 8). A significant treatment effect was also apparent in July with wool growth rate in the 9-day infused group higher than either of the other infused groups (P<0.01). This difference in growth rate persisted until November.

Over the first 2 months of the experiment the mean fibre diameter of wool from the 9-day group did not differ from the mean fibre diameters of the other prolactin infused groups. However, during June a treatment effect was evident, with the 9-day mean fibre diameter significantly higher than the 3-day infused group (FIG. 8; P<0.01).

In July, the mean fibre diameter of the 9-day infused group was approximately 3 μm greater than the other infused groups (3-day, P<0.01; 18-day, P<0.025). Significant mean fibre diameter differences persisted until October. Total cumulative patch clean wool production over the 12-month duration of the trial differed significantly between treatment groups (FIG. 8). Mean total patch weight for the 9-day infused ewes was greater than that of 3-day infused ewes (0.91 vs. 0.74 g, P<0.025) and of the 18-day infused ewes (0.91 vs. 0.66 g, P<0.01).

Prolactin receptor gene expression dropped significantly in all groups except the 3-day group following the commencement of the prolactin infusions (FIG. 9). Following the termination of the infusions, prolactin receptor gene expression rose in all infused groups although this was not statistically significant.

Trial 5

Effect of Two Separate Periods of Prolactin Treatment on Plasma Prolactin and Prolactin Receptor Gene Expression in Non-Pregnant Ewes

Trial 5 was conducted to examine two separated periods of exogenous prolactin administration on prolactin receptor gene expression in sheep skin over spring and early summer.

Twenty-one, mixed age, non-pregnant Romney ewes were maintained indoors in individual pens from September 2001 until February 2002. The ewes were fed to maintain a constant live weight. They were divided into 3 groups: short day controls (n=7), short day prolactin infused (n=7) and normal day controls (n=7). An ovine prolactin pituitary extract (1.0 mg/kg/day) was administered to each ewe in the short day infused group by intravenous infusion via a jugular cannula commencing on 9 October for 9 days. A second 9-day prolactin infusion was administered to the same ewes commencing on 20 November, 33 days after the end of the first infusion.

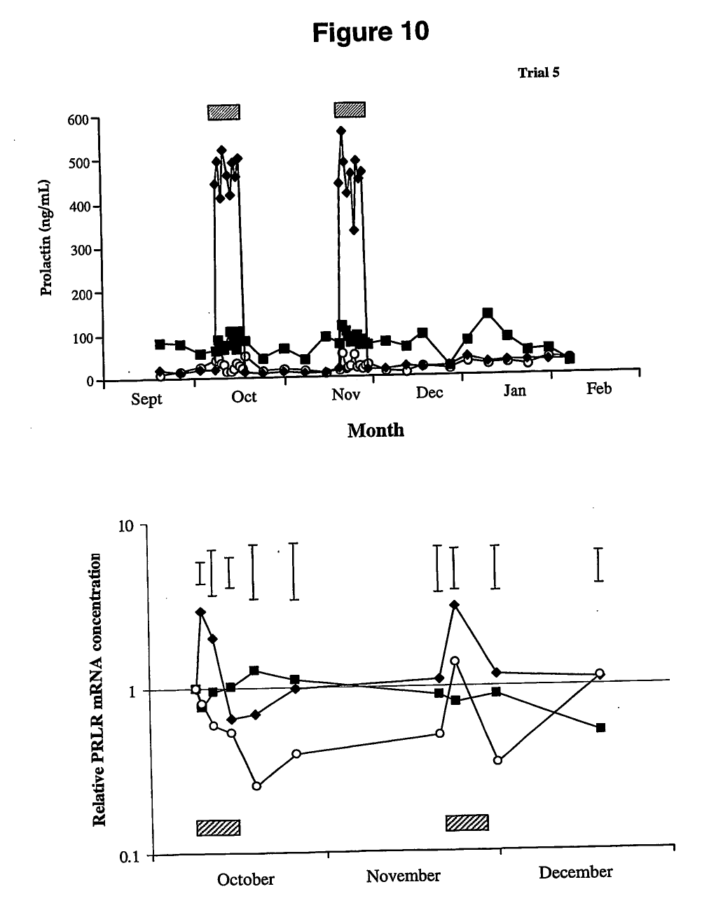

Plasma prolactin concentrations were measured at regular intervals and ten midside skin biopsies were collected at intervals between October and December 2001 for measurement of prolactin receptor long form gene expression by real-time PCR. The results are plotted in FIG. 10.

Results

In the absence of exogenous administration, prolactin concentrations remained low in both short day groups, but were elevated in the natural day group due to exposure to long day late-spring and early-summer photoperiod (FIG. 10). Intravenous prolactin increased prolactin levels in treated ewes to approximately 500 ng/ml (P<0.001), which fell rapidly to baselines levels on termination of the infusions. Prolactin receptor gene expression was elevated by prolactin at both the first (P<0.01) and at the second (P<0.02) infusions.

Trial 6

Effect of Prolactin Infusion on Prolactin Receptor Gene Expression in Pregnant Ewes of Two Different Sheep Breeds

Trial 6 was conducted to examine the effects of exogenous prolactin administration on prolactin receptor gene expression in pregnant and lactating ewes of two different sheep breeds.

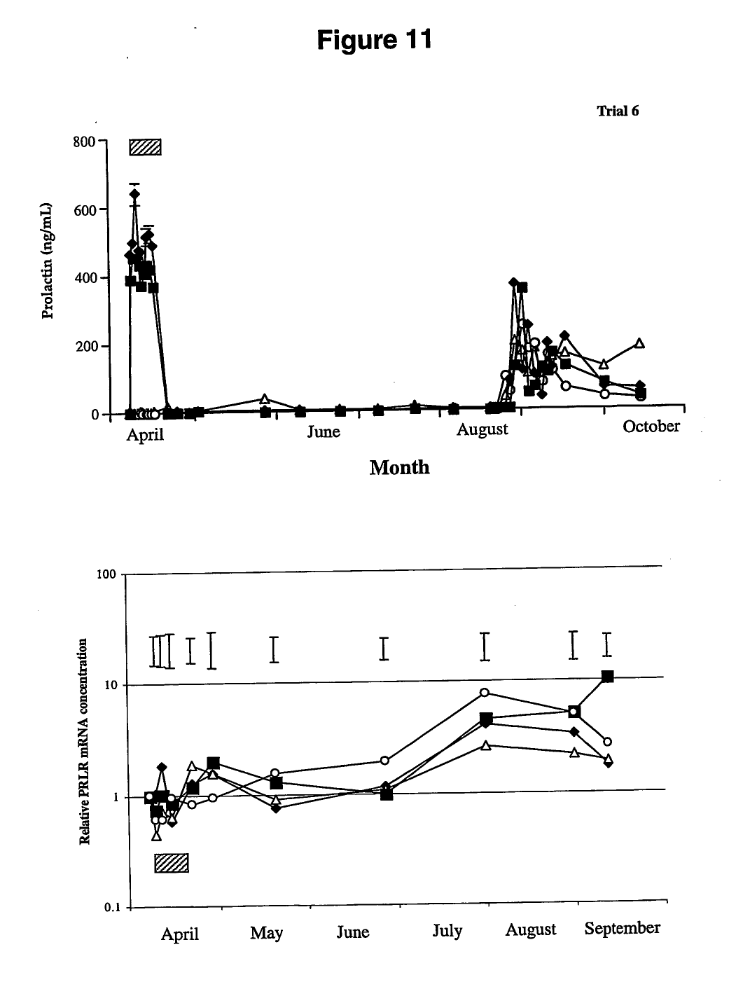

Four groups, each comprised of seven 1-2 year old Romney or Wiltshire ewes were shorn and maintained indoors in individual pens from March until November 2002. The animals were fed to maintain a constant maternal live weight (independent of conceptus and fleece weights). The ewes were mated in March, and maintained in short days (8L:16D). Half of the ewes were cannulated and infusions of prolactin (40 mg/day) for 9 days commenced one week after mating. Blood samples and skin biopsies were collected from all ewes at intervals to monitor plasma prolactin and skin prolactin receptor gene expression respectively. The results are plotted in FIG. 11.

Results

Five ewes failed to become pregnant and their data are excluded from the analysis.

Until close to parturition, prolactin levels remained low except during prolactin administration when levels in infused ewes increased to approximately 500 ng/ml (P<0.001). On termination of the infusions, prolactin concentrations shortly before parturition, circulating prolactin increased and remained elevated during lactation in all ewes (P<0.001). Prolactin receptor gene expression was higher in Wiltshire than in Romney ewes for the 5 skin samples collected between May and September (P<0.05). Prolactin treatment of Wiltshire ewes in April was associated with an increase in prolactin receptor gene expression in September (P<0.05) compared with non-treated Wiltshire ewes.

Trial 7

Effect of Prolactin Infusion on Prolactin Receptor Gene Expression in Rabbit Tissues

The effect of exogenous ovine prolactin administration on prolactin receptor gene expression in three different rabbit tissues was measured.

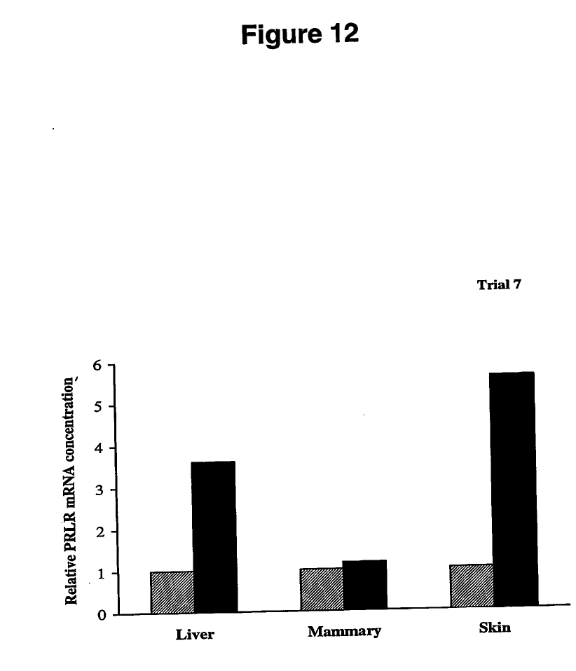

Two New Zealand White rabbits were maintained in cages indoors under ambient conditions and, fed a diet of formulated rabbit pellets ad libitum with access to fresh tap water. At 9 months of age, subcutaneous implants releasing 1.67 mg/day of bromocriptine over 60 days (Cat, Number SC-231; Innovative Research of America Inc.) were inserted under anaesthetic. At the same time, the rabbits received a slow release 2 ml osmotic pump (Alzet Model 2ML1; Alza Corporation, Palo Alto, Calif., USA) delivering either saline or ovine prolactin (1 mg/kg/day). The rabbits were blood sampled from the ear on Days 0, 3 and 7. The blood was centrifuged and the plasma stored at −20 C. until radioimmunoassay for ovine prolactin. At Day 7, the rabbits were euthanased with sodium pentobarbitone. Liver, mammary gland and skin were dissected from each rabbit and snap frozen in liquid nitrogen prior to assay by real-time PCR for prolactin receptor gene expression. The results are plotted in FIG. 12.

Results

Prolactin treatment was associated with elevated prolactin gene expression in liver and skin, but not mammary gland, at 7 days (FIG. 12).

Trial 8

Effect of Prolactin Infusion on Prolactin Receptor Gene Expression in Mice Tissues

The effect of two exogenous ovine prolactin treatments on prolactin receptor gene expression in mice mammary gland was examined.

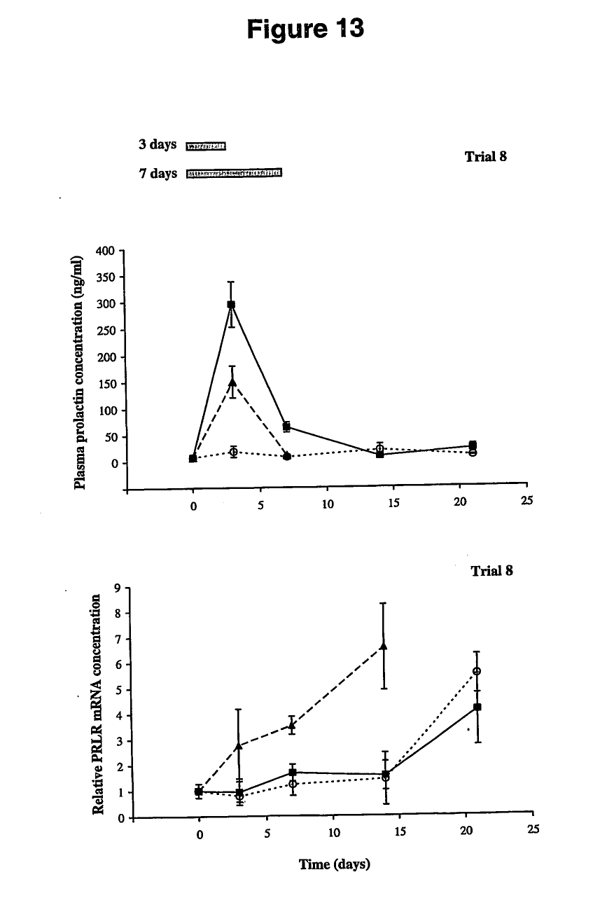

Swiss female mice were maintained in cages at a constant temperature of 22 C., under a photoperiod regime of 14 hours light: 10 hours dark, fed a diet of formulated mouse pellets ad libitum with access to fresh tap water. At 21 days of age, subcutaneous implants releasing 250 μg/day of bromocriptine over 60 days (Cat. Number SC-231; Innovative Research of America Inc.) were inserted under anaesthetic. At the same time, the mice received a slow release 100 μl osmotic pump (Alzet Model 1007D; Alza Corporation, Palo Alto, Calif.) delivering ovine prolactin (400 μg/day) for either 3 or 7 days. Mice were sacrificed in groups of 3 at intervals before, during and after prolactin administration. They were anaesthetised using CO₂ and blood sampled by heart puncture, prior to euthanasia by cervical dislocation. The blood was centrifuged and the plasma stored at −20 C. by radioimmunoassay for ovine prolactin. The mammary glands were dissected from each mouse and snap frozen in liquid nitrogen prior to assay by real-time PCR for prolactin receptor gene expression. The results are plotted in FIG. 13.

Results

Administration of ovine prolactin via osmotic pumps produced circulating levels in mice of 149 ng/ml (3-day pumps) and 294 ng/ml (7 day pumps). These differences were significant compared with saline treated controls (P<0.0.02, 3-day pumps; P<0.02, 7-day pumps). Prolactin receptor gene expression was elevated in the 3-day infused group compared to both the control group and the 7-day infused group at days 7 and 14 (P<0.05).

Discussion of Trials

Long-Term Regulation of Prolactin Receptor Gene Transcription by Prolactin

The inventors have demonstrated that expression of the prolactin receptor gene is regulated in the skin over a period of 3 months in response to a hormonal stimulus.

Earlier studies showed that changes in circulating prolactin could trigger hair cycle progression [Dicks et al., 1994; Pearson et al., 1993; Pearson et al., 1999a]. The close association between the level of prolactin receptor mRNA in the skin and follicle growth status suggests that cellular activity in the follicle is related to receptor abundance and the consequent level of signalling. Hence, prolactin may function not only to bind and activate its receptors but also to contribute to regulation of those receptors.

Both down- and up-regulation of prolactin receptor were evident in ovine skin over the 30 day period of steadily increasing plasma prolactin. During the first seven days of the induced wool growth cycle in Wiltshire sheep (Trial 1), there was an initial decline in the abundance of prolactin receptor mRNA associated with an increase in circulating prolactin.

As plasma prolactin continued to increase, prolactin receptor regulation was reversed and mRNA became more abundant in the skin. The concurrence of peak prolactin receptor expression and high circulating prolactin with the initiation of follicle growth suggests a stimulatory role in follicle recrudescence, as in mammary and reproductive tissues [Cassy et al., 1998].

Over short periods of time, such influences of prolactin on its own receptor are well recognised. Indeed, simultaneous and opposite prolactin receptor changes have been shown in different organs of rats infused with ovine prolactin and growth hormone, depending on reproductive status and lactogen concentration [Barash et al., 1986].

Rapid “down-regulation” of binding sites has been attributed to an increase in the rate of receptor internalisation and degradation [Djiane et al., 1979; Djiane et al., 1982]. However, long-term up-regulation in a seasonal animal in response to a prolactin surge has not previously been demonstrated.

This allows for the possibility of enhancing or inhibiting physiological responses to prolactin by appropriate priming treatments that change prolactin receptor numbers in the target tissue. Such effects are demonstrated in Trials 2 and 3 where large changes in wool growth were measured in response to alterations of prolactin status in prior months. In Trial 2, this could be seen in response to increased prolactin over pregnancy, parturition and lactation associated with long day photoperiod. In Trial 3, an 18-day infusion of prolactin immediately after mating was associated with increased wool production during pregnancy and after lambing.

Such long-term effects are also demonstrated in Trials 2-6 and in Trial 8 where increased levels of prolactin receptor mRNA were observed during and after a short-term prolactin treatment. Further, these effects are dependent on the prolactin profile (Trial 3), duration of infusion (Trial 4), timing of the infusion (Trial 5) and genotype (Trial 6). Treatments to alter future physiological responses to prolactin will vary with genotype, species and tissue (Trials 6, 7 and 8) but can be assessed on the basis of similar trials to determine prolactin receptor responses to profiles of exogenous prolactin or of prolactin mimetics. To assist in such assessments, the inventors have developed a mathematical model of the prolactin signalling system as described in the following section.

Mathematical Model Including Equations and Diagram

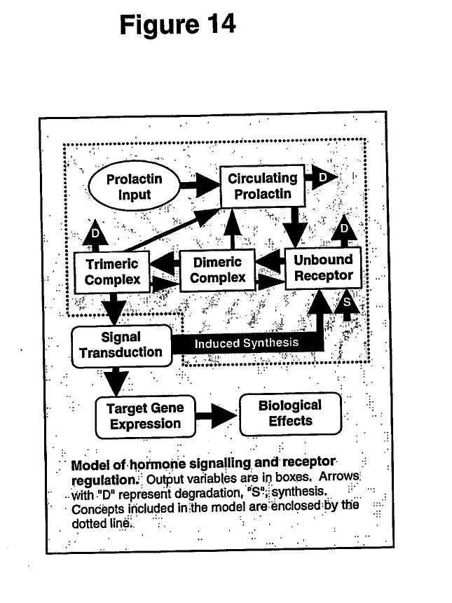

The model structure is represented in FIG. 14. The variables of interest are the concentration of plasma prolactin and the numbers of unbound prolactin receptor, hormone-receptor dimers and hormone-receptor trimers at any time, t. Prolactin binding is sequential. First, the hormone interacts with its receptor through one binding site forming an inactive hormone-ligand complex. Then, prolactin binds to a second receptor, which leads to formation of signalling trimeric complex consisting of a prolactin molecule and its two receptors. We assume that the biological effect of the hormone is a function of the trimer concentration.

In the model, the number of bound receptors increases due to binding of prolactin to unbound receptors and decreases because of degradation and dissociation back to the unbound state. We assume the rate of binding and dissociation obey a mass-action law and that the degradation of receptors is represented as a first order process.

Denoting the number of receptors in dimer and trimer complexes by B₁(t) and B₂(t) respectively, the number of unbound receptors by U(t) and the plasma prolactin concentration by P(t), we express this statement mathematically by the differential equation

$$\frac{ⅆ {B}_{1}}{ⅆ t} = α P ( t ) U ( t ) - {α}_{1} {B}_{1} ( t ) U ( t ) - d {B}_{1} ( t ) + {d}_{12} {B}_{2} ( t ) - {δ}_{1 b} {B}_{1} ( t )$$

$$\frac{ⅆ {B}_{2}}{ⅆ t} = {α}_{1} {B}_{1} ( t ) U ( t ) - {d}_{12} {B}_{2} ( t ) - {δ}_{2 b} B ( t )$$

Here α and α₁ are rates of formation of dimer and trimer complexes respectively and d, δ_{1b}, d₁₂, δ_{2b} are the dissociation and degradation rates of hormone-receptor complexes respectively.

The number of unbound receptors decreases because of binding and degradation and increases due to synthesis and dissociation processes. We separate receptor synthesis at a constant rate, α, from induced synthesis with the rates depending on the size of the pool of signalling trimer complex. In the current model, we take this dependence on B₂(t) to be Michaelis-Menten. Then it is linear for small values and saturating for large values of B(t). The corresponding differential equation for number of unbound receptors is

$$\frac{ⅆ U}{ⅆ t} = a - {δ}_{u} U ( t ) - α P ( t ) U ( t ) - {α}_{1} {B}_{1} ( t ) U ( t ) + d {B}_{1} ( t ) + {d}_{12} {B}_{2} ( t ) + \frac{μ {B}_{2} ( t )}{{b}_{0} + {B}_{2} ( t )}$$

The parameters μ and b₀ are associated with induced synthesis, with μ being the maximum rate of induced synthesis and b₀ the number of receptors bound in trimer complexes at which the rate of induced synthesis is half the maximum value. The parameter δᵤ is associated with the natural protein degradation.

The concentration of plasma prolactin decreases because of clearance, whilst secretion and artificial prolactin input cause an increase. These terms have already been described in the previous equations so the differential equation for concentration of plasma prolactin is

$$\frac{ⅆ P}{ⅆ t} = - γ P ( t ) + {k}_{0} + {k}_{1} ( t )$$

where γ is the clearance rate constant, k₀ and k(t) describe the secretion and external input of prolactin respectively.

It is possible to parameterise the model for a particular species or tissue by setting the appropriate variables in the model to design the optimal timing, plasma profile and dosage of a temporary sustained prolactin (or a mimetic) treatment or immunological manipulation to alter short and long-term physiological responses to these.

For the simulations shown below we have taken values for parameters from the range currently available in literature [Gertler et al., 1996]
α=4.15 (litres·nmol⁻¹·day⁻¹), α₁=
3.02 (litres·nmol⁻¹·day⁻¹), d=12.96 (day⁻¹), d₁₂=4717.4 (day⁻¹), b₀=125000 (nmol), γ=35.6 (day⁻¹), μ=310000, δ_{1b}=δ_{b}=0.75 (day⁻¹), δᵤ=0.53 (day⁻¹), and α=0.827.2 (nmol/day). At this stage, we ignore seasonal and animal physiological effects and take prolactin secretion at the constant rate k₁=0.39 (nmol/day) in accordance with experimental results for sheep held in short days [Pearson et al., 1996; Nixon et al., 2002].

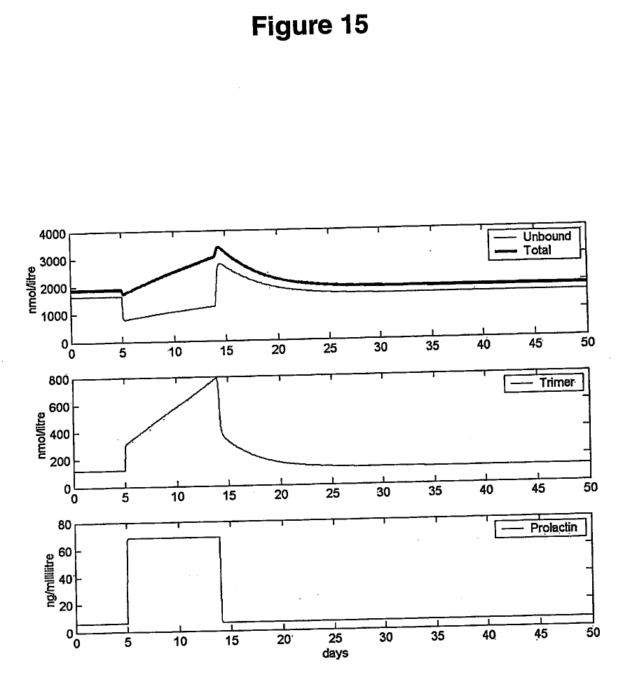

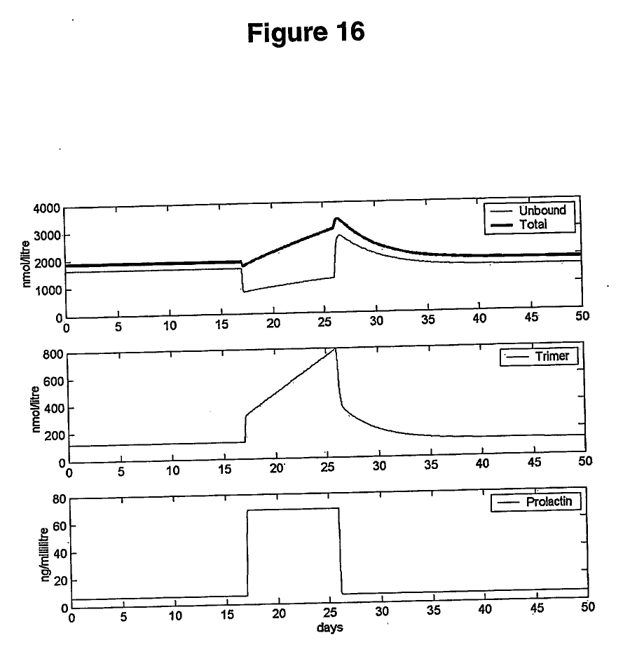

Initial conditions are determined by equilibrium under the above conditions. The number of unbound receptors drops as the prolactin is infused, with a corresponding rise in bound receptor number. The induced synthesis of unbound receptors due to the presence of bound receptors (positive up-regulation) halts the decline, and once the infusion is switched off the number of bound receptors declines back to the initial equilibrium, the number of unbound receptors reaches their highest level and then relaxes to the equilibrium level (FIGS. 15 and 16).

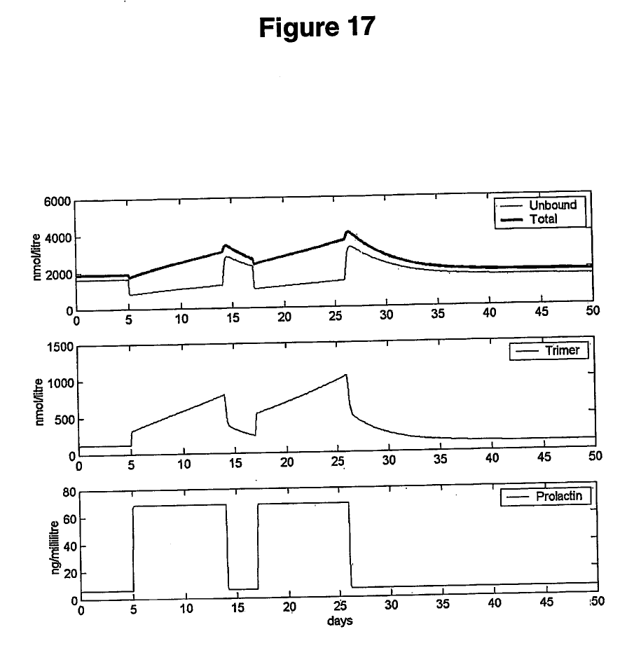

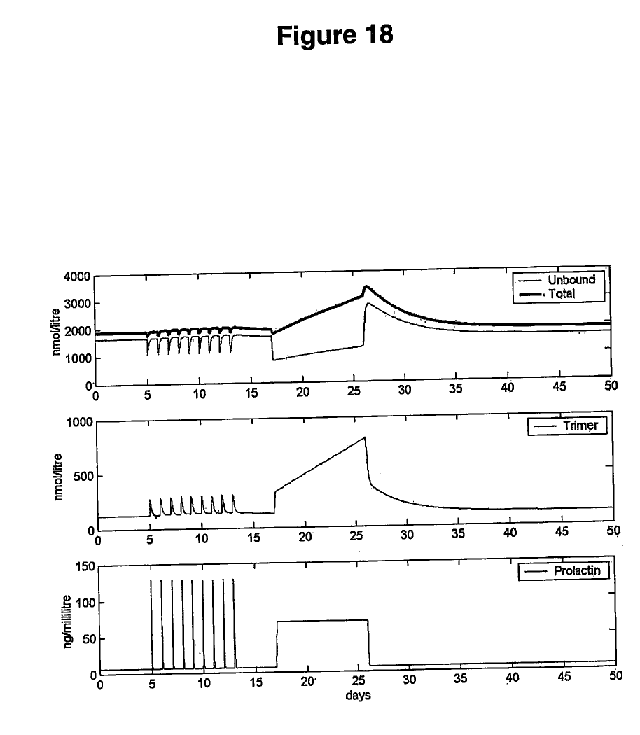

FIG. 17 shows the simulation of two consequent nine-day prolactin infusions. FIG. 18 shows the comparison of effects of infusion and administration of the same amount of prolactin by series of injections.

Discussion of Model

The inventors have devised the model to predict the effect of a particular treatment as a guideline for developing the best method for treatment or of experimental design.

Using this model, it is possible to parameterise for a particular species or tissue and design the optimal timing, plasma profile and dosage of a temporary sustained prolactin (or a mimetic) treatment or immunological manipulation to alter short and long-term physiological responses to these. The effect of prolactin treatment strongly depends on parameters of the model. These can vary significantly for different tissues and species.

The five model simulations presented in FIGS. 15-19 illustrate how the invention can be utilised and show how prolactin treatment in sheep (in these examples presented as 9 day infusions or a series of 9 daily injections of exogenous prolactin) predicts short and long-term changes in the number of bound and unbound prolactin receptors. The simulations show:

Simulation 1 (FIG. 15): A single prolactin fusion of 9 days duration commencing at day 5 causes an initial decrease in the number of unbound receptors followed by a up-regulation in unbound receptor numbers (and therefore increases potential capacity to respond to subsequent prolactin or prolactin mimetic stimulation). The up-regulation of unbound receptors is in accord with the mRNA data from Trial 1 (FIG. 2).

Simulation 2 (FIG. 16): A single prolactin infusion commencing at day 17 causes an initial decrease in the number of unbound receptors followed by a relatively long-term up-regulation in unbound receptor numbers, but displaced with respect to Simulation 1.

Simulation 3 (FIG. 17): An initial prolactin (or prolactin mimetic) treatment commencing at day 5 results in an augmentation of unbound receptors at the time of a second treatment commencing at day 17 (and therefore predicts an increase in any physiological response at that time). This is consistent with the wool data presented in Trials 2 and 3 (FIGS. 3 and 4).

Simulation 4 (FIG. 18): The effect of prolactin administration by infusion is compared with the effect of series of prolactin injections. The results are consistent with Trial 3 (FIG. 4).

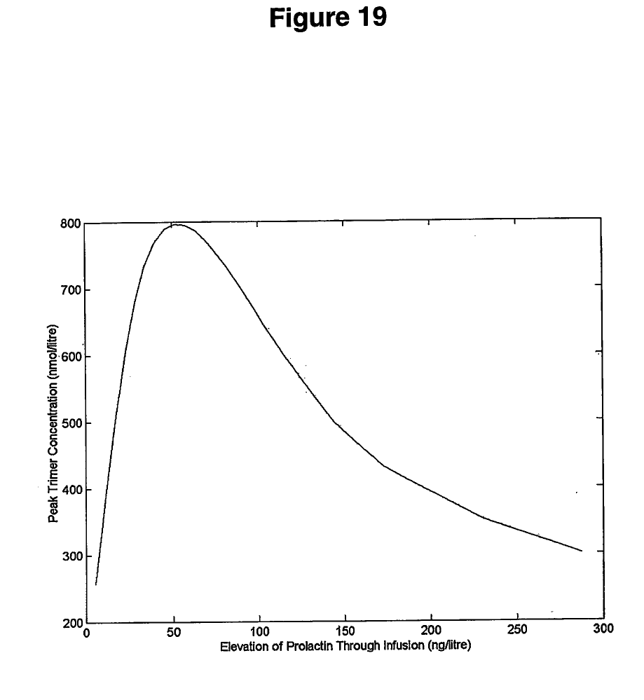

Simulation 5 (FIG. 19): The effect of prolactin infusion is sensitive to the duration and amplitude of the injection. For the 9 days infusion, the concentrations of bound receptors were simulated over a wide range of infused hormone concentrations. Trimer concentration first increases with hormone concentration until a maximum is reached, then decreases continuously at higher prolactin concentrations. For the chosen parameterisation and duration of infusion, a maximum response is reached for a ten-fold increase in prolactin level. This value can be shifted to higher concentrations of prolactin by increasing the infusion duration. The results are consistent with Trial 8 (FIG. 13).

Aspects of the present invention have been described by way of example only and it should be appreciated that modifications and additions may be made thereto without departing from the scope thereof.

### REFERENCES

Akers R M, Bauman D E, Capuco A V, Goodman G T, Tucker H A (1981): Prolactin regulation of milk secretion and biochemical differentiation of mammary epithelial cells in periparturient cows. Endocrinology 109:23-30.
Anthony R V, Smith G W, Duong A, Pratt S L, Smith M F (1995): Two forms of the prolactin receptor messenger ribonucleic acid are present in ovine fetal liver and adult ovary. Endocrine 3:291-295.
Aston R, Holder A T, Rathjen D A, Trigg T E, Moss B A, Pell J A (1991): Antibody mediated enhancement of growth hormone activity: application to animal production. Proceedings of the New Zealand Society of Animal Production 51:227-234.
Auldist M J, Walsh B J, Thomson N A (1998): Seasonal and lactational influences on bovine milk composition in New Zealand. Journal of Dairy Research 65:401-411.
Barash I, Madar Z, Gertler A (1983): Down regulation of prolactin receptors in the liver, mammary gland and kidney of female virgin rat infused with ovine prolactin or human growth hormone. Biochemical and Biophysical Research Communications 116:644-650.
Barash I, Madar Z, Gertler A (1986): Short-term regulation of prolactin receptors in the liver, mammary gland and kidney of pregnant and lactating rats infused with ovine prolactin or human growth hormone. Molecular and Cellular Endocrinology 46:235-244.
Barenton B, Pelletier J (1980): Prolactin, testicular growth and LH receptors in the ram following light and 2-Br-alpha-ergocryptine (CB-154) treatments. Biology of Reproduction 22:781-790.
Barrington G M, Besser T E, Gay C C, Davis W C, Reeves J J, McFadden T B (1997): Effect of prolactin on in vitro expression of the bovine mammary immunoglobulin G1 receptor. Journal of Dairy Science 80:94-100.
Beattie J, Holder A T (1994): Location of an epitope defined by an enhancing monoclonal antibody to growth hormone: some structural details and biological implications. Molecular Endocrinology 8:1103-1110.
Bole-Feysot C, Goffin V, Edery M, Binart N, Kelly P A (1998): Prolactin (PRL) and its receptor: Actions, signal transduction pathways and phenotypes observed in PRL receptor knockout mice. Endocrine Reviews 19:225-268.
Brinklow B R, Forbes J M (1983): Prolactin infusion causes increased nitrogen retention in lambs in continuous darkness. Proceedings of the Nutrition Society 42:38A.
Cassy S, Charlier M, Belair L, Guillomot M, Charron G, Bloch B, Djiane J (1998): Developmental expression and localization of the prolactin receptor (PRL-R) gene in ewe mammary gland during pregnancy and lactation: Estimation of the ratio of the two forms of the PRL-R messenger ribonucleic acid. Biology of Reproduction 58:1290-1296.
Cassy S, Charlier M, Guillomot M, Pessemesse L, Djiane J (1999): Cellular localization and evolution of prolactin receptor mRNA in ovine endometrium during pregnancy. FEBS Letters 445:207-211.
Choy V J, Nixon A J, Pearson A J (1997): Distribution of prolactin receptor immunoreactivity in ovine skin and during the wool follicle growth cycle. Journal of Endocrinology 155:265-275.
Craven A J, Ormandy C J, Robertson F G, Wilkins R J, Kelly P A, Nixon A J, Pearson A J (2001) Prolactin signaling influences the timing mechanism of the hair follicle: analysis of hair growth cycles in prolactin receptor knockout mice. Endocrinology 142:2533-2539.
Curlewis J D (1992): Seasonal prolactin secretion and its role in seasonal reproduction: a review. Reproduction, Fertility and Development 4:1-23.
Dahl G E, Elsasser T H, Capuco A V, Erdman R A, Peters R R (1997): Effects of a long daily photoperiod on milk yield and circulating concentrations of insulin-like growth factor-1. Journal of Dairy Science 80:2784-9.
Dicks P, Russel A J F, Lincoln G A (1994): The role of prolactin in the reactivation of hair follicles in relation to moulting in cashmere goats. Journal of Endocrinology 143:441-448.
Djiane J, Dusanter Fourt I, Katoh M, Kelly P A (1985): Biological activities of binding site specific monoclonal antibodies to prolactin receptors of rabbit mammary gland. Journal of Biological Chemistry 260:11430-11435.
Djiane J, Clauser H, Kelly P A (1979): Rapid down-regulation of prolactin receptors in mammary gland and liver. Biochemical and Biophysical Research Communications 90:1371-1378.

Djiane J, Delouis C, Kelly P A (1982): Prolactin receptor turnover in explants of pseudopregnant rabbit mammary gland. Molecular and Cellular Endocrinology 25:163-170.
Djiane J, Kelly P A (1993): Prolactin. In Thibault C, Levasseur M C, Hunter R H F (eds): “Reproduction in mammals and man.” Paris: Ellipses, pp 121-133.
Fitzgerald B P, Evins J D, Cunningham F J (1981): Effect of TRH on the secretion of prolactin in ewes at various stages of pregnancy and in non-pregnant ewes during the breeding season and seasonal anoestrus. Journal of Reproduction and Fertility 61:149-155.
Forsyth I A, Lee P D (1993): Bromocriptine treatment of periparturient goats long-term suppression of prolactin and lack of effect on lactation. Journal of Dairy Research 60:307-317.
Freeman M E, Kanyicska B, Lerant A, Nagy G (2000): Prolactin: structure, function, and regulation of secretion. Physiological Reviews 80:1523-1631.
Freemark M, Driscoll P, Maaskant R, Petryk A & Kelly P A (1997) Ontogenesis of prolactin receptors in the human fetus in early gestation. Implications for tissue differentiation and development. J Clin Invest 99: 1107-1117.
Fulkerson W J, McDowell G H, Fell L R (1975): Artificial induction of lactation in ewes: the role of prolactin. Australian Journal of Bological Science 28:525-530.
Gertler A, Grosclaude J, Strasburger C J, Nir S, Djiane J (1996): Real time kinetic measurement of the interaction between lactogenic hormones and prolactin-receptor extracellular domains from several species support the model of hormone-induced transient receptor dimerisation. Journal of Biological Chemistry 271:24482-24491.
Goffin V, Binart N, Touraine P, Kelly P A (2002): Prolactin: the new biology of an old hormone. Annual Review of Physiology 64:47-67.
Grasselli F, Gaiani R, Tamanini C (1992): Seasonal variation in the reproductive hormones of male goats. Acta Endocrinologica 126:271-275.
Hansen P J (1985): Seasonal modulation of puberty and the post-partum anestrus in cattle: a review. Livestock Production Science 12:309-327.
Hawker H, Crosbie S F, Thompson K F, McEwan J C (1984): Effects of season on the wool growth response of Romney ewes to pasture allowance. Animal Production in Australia 15:380-383.
Hill R A, Pell J M (1998): Regulation of insulin like growth factor I (IGF I) bioactivity in vivo: Further characterization of an IGF I enhancing antibody. Endocrinology 139:1278-1287.
Hogan B, Beddington R, Costantini F, Lacy E (1994) In: Manipulating the mouse embryo: A laboratory manual, Second Edition, Cold Spring Harbour Laboratory Press (Cold Spring Harbor, N.Y.), 487 pp.
Holder A T, Aston R, Preece M A, Ivanyi J (1985): Monoclonal antibody mediated enhancement of growth hormone activity in vivo. Journal of Endocrinology 107:R9-R12.
   Johke T, Hodate K (1983): Circulating prolactin leve and lactogenesis in dairy cows. Proceedings of the Fifth World Congress on Animal Production 2:251-252.
Kelly P A, Robertson H A, Friesen H G (1974): Temporal pattern of placental lactogen and progesterone secretion in sheep. Nature 248:435-437.
Kendall P E (1999): Prolactin and wool growth in the Romney ewe: Ph.D. Thesis. Palmerston North, NZ: Massey University.
Lamming G E, Moseley S R, McNeilly J R (1974): Prolactin release in the sheep. Journal of Reproduction and Fertility 40:151-168.
L'Huillier P J, Soulier S, Stinnakre M-G, Lepourry L, Davis S R, Mercier J-C, Vilotte J-L (1996): Efficient and specific ribozyme-mediated reduction of bovine beta-lactalbumin expression in double transgenic mice. Proceedings of the National Academy of Sciences 93:6698-6703.
Lincoln G A (1989): Significance of seasonal cycles in prolactin secretion in male mammals. Perspectives in Andrology 53:299-306.
Lincoln G A (1990): Correlation with changes in horns and pelage, but not reproduction, of seasonal cycles in the secretion of prolactin in rams of wild, feral and domesticated breeds of sheep. Journal of Reproduction and Fertility 90:285-296.
Lincoln G A, Ebling F J P (1985): Effect of constant-release implants of melatonin on seasonal cycles in reproduction, prolactin secretion and moulting in rams. Journal of Reproduction and Fertility 73:241-253.
Ling J K (1970): Pelage and molting in wild mammals with special reference to aquatic forms. Quarterly Review of Biology 45:16-54.
Loudon A S I, Brinklow B R (1990): Melatonin implants prevent the onset of seasonal quiescence and suppress the release of prolactin in response to a dopamine antagonist in the Bennett's wallaby (Macopus rufogriseus rufogriseus). Journal of Reproduction and Fertility 90:611-618.
Maaskant R A, Bogic L V, Gilger S, Kelly P A & Bryant-Greenwood G D (1996) The human prolactin receptor in the fetal membranes, decidua, and placenta. J Clin Endocrinol Metab 81: 396-405.
Maes M, Mommen K, Hendrickx D, Peeters D, D'Hondt P, Ranjan R, De Meyer F, Scharpé S (1997): Components of biological variation, including seasonality, in blood concentrations of TSH, TT3, FT4, PRL, cortisol and testosterone in healthy volunteers. Clinical Endocrinology (Oxford) 46:597-598.
Martinet L, Ravault J P, Meunier M (1982): Seasonal variations in mink (Mustela vison) plasma prolactin measured by heterologous radioimmunoassay. General and Comparative Endocrinology 48:71-75.
Masters D G, Stewart C A, Connell P J (1993): Changes in plasma amino acid patterns and wool growth during late pregnancy and early lactation in the ewe. Australian Journal of Agricultural Research 44:945-957.
Mena F, Martinez-Escalera G, Aguayo D, Clapp C, Grosvenor C E (1982): Latency and duration of the effects of bromocriptine and prolactin on milk secretion in lactating rabbits. Journal of Endocrinology 94:389-95.
Michael S D (1976): Plasma prolactin and progesterone during the estrous cycle in the mouse. Proceedings of the Society for Experimental Biology and Medicine 153:254-257.
Mondain-Monval M, Møller O M, Smith A J, McNeilly A S, Scholler R (1985): Seasonal variations of plasma prolactin and LH concentrations in the female blue fox (Alopex lagopus). Journal of Reproduction and Fertility 74:439-448.
Newbold J A, Chapin L T, Zinn S A, Tucker H A (1991): Effects of photoperiod on mammary development and concentration of hormones in serum of pregnant dairy heifers. Journal of Dairy Science 74:100-108.
Nicoll C S (1980): Ontogeny and evolution of prolactin's functions. Federation Proceedings 39:2563-2566.
Nixon A J (1993): A method for determining the activity state of hair follicles. Biotechnic and Histochemistry 68:316-325.
Nixon A J, Choy V J, Parry A L, Pearson A J (1993): Fibre growth initiation in hair follicles of goats treated with melatonin. Journal of Experimental Zoology 267:47-56.
Nixon A J, Ford C A, Oldham J M, Pearson A J (1997): Localization of insulin-like growth factor receptors in skin follicles of sheep (Ovis aries) and changes during an induced growth cycle. Comparative Biochemistry and Physiology Part A 118A:1247-1257.
Nixon A J, Ford C A, Wildermoth J E, Craven A J, Pearson A J (2002): Regulation of prolactin receptor expression in ovine skin in relation to circulating prolactin and wool follicle growth status. Journal of Endocrinology 172:605-614.
Oddy V H (1985): Wool growth of pregnant and lactating Merino ewes. Journal of Agricultural Science 105:613-622.
Ohlson D L, Spicer L J, Davis S L (1981): Use of active immunisation against prolactin to study the influence of prolactin on growth and reproduction in the ram. Journal of Animal Science 52:1350-1359.
Ormandy C J, Binart N, Helloco C, Kelly, P A (1998): Mouse prolactin receptor gene: genomic organization reveals alternative promoter usage and generation of isoforms via alternative 3′-exon splicing. DNA & Cell Biology. 17 (9): 761-770.
Ostrom K M (1990): A review of the hormone prolactin during lactation. Progress in Food and Nutrition Science 14:143.
Parry A L, Nixon A J, Craven A J, Pearson A J (1996): The microanatomy, cell replication, and keratin gene expression of hair follicles during a photoperiod-induced growth cycle in sheep. Acta Anatomica 154:283-299.
Pearson A J, Ashby M G, Choy V J, Nixon A J, Wildermoth J E (1993): The effects on wool follicle growth of suppression of the plasma prolactin surge following short to long day transition. Journal of Endocrinology 139:Abstract P46.
Pearson A J, Ashby M G, Wildermoth J E, Craven A J, Nixon A J (1999a): Effect of exogenous prolactin on the hair growth cycle. Experimental Dermatology 8:358-360.
Pearson A J, Kendall P E, Ashby M G, Wildermoth J E (1999b): Fleece production patterns in Romney ewes: effects of photoperiod, pregnancy and lactation. Proceedings of the New Zealand Society of Animal Production 59:30-33.
Pearson A J, Parry A L, Ashby M G, Choy V J, Wildermoth J E, Craven A J (1996): Inhibitory effect of increased photoperiod on wool follicle growth. Journal of Endocrinology 148:157-166.
Peel C J, Taylor J W, Robinson I B, McGowan A A, Hooley R D, Findlay J K (1978): The importance of prolactin and the milking stimulus in the artificial induction of lactation in cows. Australian Journal of Biological Sciences 31:187-195.
Pell J M, Aston R (1995): Principles of immunomodulation. Livestock Production Science 42:123-133.
Peters R R, Chapin L T, Leining K B, Tucker H A (1978): Supplemental lighting stimulates growth and lactation in cattle. Science 199:911-2.
Peters R R, Chaplin L T, Emery R S, Tucker H A (1980): Growth and hormonal responses to various photoperiods. Journal of Animal Science 51:1148-1153.
Peterson S W, Mackenzie D D S, McCutcheon S N (1990): Milk production and plasma prolactin levels in spring- and autumn-lambing ewes. Proceedings of the New Zealand Society of Animal Production 50:483-485.
Peterson S W, Mackenzie D D S, McCutcheon S N (1991): Effect of inhibiting prolactin secretion in ewes during late pregnancy on lactogenesis. Proceedings of the New Zealand Society of Endocrinology. Supplement to the Proceedings of the Endocrine Society of Australia 34:199.
Peterson S W, Mackenzie D D S, McCutcheon S N, Lapwood K R (1997): Long-term bromocriptine treatment during late pregnancy has differential effects on milk yields of single- and twin-bearing ewes. New Zealand Journal of Agricultural Research 40:249-259.
Petitclerc D, Vinet C, Roy G, Lacasse P (1998): Prepartum photoperiod and melatonin feeding on milk production and prolactin concentrations of dairy heifers and and cows. Journal of Dairy Science 81:251.
Posner B I, Kelly P A, Friesen H G (1975): Prolactin receptors in rat liver: possible induction by prolactin. Science 188:57-59.
Prandi A, Motta M, Chiesa F, Tamanini C (1988): Circannual rhythm of plasma prolactin concentration in the goat. Animal Reproduction Science 17:85-94.
Regisford E G, Katz L S (1993): Effects of bromocriptine-induced hypoprolactinaemia on gonadotrophin secretion and testicular function in rams (Ovis aries) during two seasons. Journal of Reproduction and Fertility 99:529-537.
Regisford E G, Katz L S (1994): Testicular LH receptors are dependent on serum prolactin (PRL) concentrations in rams. Biology of Reproduction 50:112.
Reiter R J (1980): The pineal and its hormones in the control of reproduction in mammals. Endocrine Reviews 1: 109-131.
Rhind S M, Robinson J J, Chesworth J M, Crofts R M J (1980): Effects of season, lactation and plane of nutrition on prolactin concentrations in ovine plasma and the role of prolactin in the control of ewe fertility. Journal of Reproduction and Fertility 58:145-152.
Rougeot J, Allain D, Martinet L (1984): Photoperiodic and hormonal control of seasonal coat changes in mammals with special reference to sheep and mink. Acta Zoologica Fennica 171:13-18.
Royster M, Driscoll P, Kelly P A, Freemark M (1995): The prolactin receptor in the fetal rat: cellular localization of messenger ribonucleic acid, immunoreactive protein, and ligand-binding activity and induction of expression in late gestation. Endocrinology 136: 3892-3900.
Rui H, Brekke I, Torjesen P A, Purvis K (1986): Homologous up-regulation of the prolactin receptor in rat prostatic explants. Molecular and Cellular Endocrinology 46:53-57.
Schams D, Russe I, Schallenberger E, Prokopp S, Chan J S D (1984): The role of steroid hormones, prolactin and placental lactogen on mammary gland development in ewes and heifers. Journal of Endocrinology 102:121-130.
Schanbacher B D, Crouse J D (1980): Growth and performance of growing-finishing lambs exposed to long or short photoperiods. Journal of Animal Science 51:943-948.
Smith A J, Mondain-Monval M, Møller O M, Scholler R, Hansson V (1985): Seasonal variations of LH, prolactin, androstenedione, testosterone and testicular FSH binding in the male blue fox (Alopex lagopus). Journal of Reproduction and Fertility 74:449-458.
Smith A J, Mondain-Monval M, Simon P, Andersen Berg K, Clausen O P F, Hofmo P O, Scholler R (1987): Preliminary studies of the effects of bromocriptine on testicular regression and the spring moult in a seasonal breeder, the male blue fox (Alopex lagopus). Journal of Reproduction and Fertility 81:517-524.
Soares M J, Faria T N, Roby K F (1991): Pregnancy and the prolactin family of hormones: Coordination of anterior pituitary, uterine, and placental expression. Endocrine Reviews 12:402-423.
Sumner R M W, Revfeim K J A (1973): Sources of variation and design criteria for wool fibre diameter measurements for New Zealand Romney sheep. New Zealand Journal of Agricultural Research 16:169-176.
Tucker H A, Petitclerc D, Zinn S A (1984): The influence of photoperiod on body weight gain, body composition, nutrient intake and hormone secretion. Journal of Animal Science 59:1610-1620.
Wettermann R P, Tucker H A (1974): Relationship of ambient temperature to serum prolactin in heifers. Proceedings of the Society for Experimental Biology and Medicine 146:908-911.
Wettermann R P, Tucker H A, Beck T W, Meyerhoeffer D C (1982): Influence of ambient temperature on prolactin concentrations in serum of Holstein and Brahman×Hereford heifers. Journal of Animal Science 55:391-394.
Wong C C, Döhler K-D, Atkinson M J, Geerlings H, Hesch R-D, von zur Mühlen A (1983): Circannual variations in serum concentrations of pituitary, thyroid, parathyroid, gonadal and adrenal hormones in male laboratory rats. Journal of Endocrinology 97:179-185.
Woods J L, Orwin D F G (1988): Seasonal variations in the dimensions of individual Romney wool fibres determined by rapid autoradiographic technique. New Zealand Journal of Agricultural Research 31:311-323.

Wu W X, Brooks J, Glasier A F, McNeilly A S (1995): The relationship between decidualization and prolactin mRNA and production at different stages of human pregnancy. Journal of Molecular Endocrinology 14:255-61.

TABLE 1
Plasma prolactin concentrations in selected mammals

| | | | Minimum | | Maximum | | |
|---|---|---|---|---|---|---|---|
| | | | Conc. | | Conc. | | |
| Species and Breed | | Sex | (ng/mL) | Season | (ng/mL) | Season | Reference |
| Mink | | M | 4 | late autumn to mid-winter | 23 | late spring | [Martinet et al., 1982] |
| | | F | 6 | late autumn to mid-winter | 36 | mid-spring | [Martinet et al., 1982] |
| Blue fox | | M | <3 | late summer to early spring | 8 | late spring | [Smith et al., 1985] |
| | | F | <3 | mid-summer to early spring | 10 | early summer | [Mondain-Monval et al., 1985] |
| Goat | | M | <2 | mid-winter | 80 | mid-summer | [Grasselli et al., 1992] |
| | | F | <20 | mid-winter | >300 | mid-summer | [Prandi et al., 1988] |
| Sheep | Merino | M | <5 | mid-winter | 165 | late spring | [Lincoln, 1990] |
| | Romney | F | <10 | mid-winter | 200 | early summer | [Kendall, 1999] |
| | Wiltshire | F | <2 | mid-winter | 150 | late spring | [Pearson et al., 1996] |

| | | | |
|---|---|---|---|
| Mouse* | F | ˜50 | [Michael, 1976] |
| Rat* | M | <20 | [Wong et al., 1983] |
| Human* | M | ˜11 | [Maes et al., 1997] |
| | F | ˜24 | [Maes et al., 1997] |

*non-seasonal, average value

TABLE 2

Oligonucleotide primers and probes used in PCR amplification of prolactin
receptor cDNA. A common forward primer was used in combination with
different reverse primers to detect different prolactin receptor isoforms.
Probes were labelled with a FAM reporter dye and a TAMRA quencher,

according to the Taqman real-time PCR system (Applied Biosystems)

| | | Amplicon size |
|---|---|---|
| Oligonucleotide identity | Primer sequence | (bp) |
| Mouse | | |
| mPRLR common forward | 5′-ATAAAAGGATTTGATACTCATCTGCTAGAG-3′ | |
| mPRLR long form reverse | 5′-TGTCATCCACTTCCAAGAACTCC-3′ | 133 |
| mPRLR short form 1 reverse | 5′-CATAAAAACTCAGTTGTTGGAATCTTCA-3′ | 92 |
| mPRLR short form 2 reverse | 5′-GGAAAAAGACATGGCAGAAACC-3′ | 113 |
| mPRLR short form 3 reverse | 5′-AGTTCCCCTTCATTGTCCAGTTT-3′ | 113 |
| mPRLR probe | 5′-CCCCCACTTCTGACTGTAGGACTTGC-3′ | |
| Sheep | | |
| oPRLR forward | 5′-GCATGGTGACCTGCATCCT-3′ | |
| oPRLR long form reverse | 5′-CGGCTTGCCCTTCTCCA-3′ | |
| oPRLR probe | 5′-CCACCAGTTCCAGGGCCAAAAAG-3′ | |
| Rabbit | | |
| rbPRLR forward | 5′-GCAGTGGCTTTGAAGGGCTAT-3′ | |
| rbPRLR reverse | 5′-CCCAGGAACTGGTGGAAAGA-3′ | 57 |
| rbPRLR probe (minor groove | 5′-CATGGTGACCTGC-3′ | |
| binding design) | | |

1-28. (canceled)

29. A method for affecting a physiological response of an animal to circulating levels of prolactin and/or prolactin mimetics, which comprises the step of:
modulating prolactin receptors by effectively signalling a sustained increase in the circulating level of prolactin and/or prolactin mimetics, followed by a decrease back to normal or low levels.

30. A method as claimed in claim 1, wherein the sustained increase in circulating level of prolactin and/or prolactin mimetics is brought about by an increase in the photoperiod to which an animal is exposed.

31. A method as claimed in claim 1, wherein the sustained increase in circulating level of prolactin and/or prolactin mimetics is brought about by intravenous infusion.

32. A method as claimed in claim 1, wherein the sustained increase in circulating level of prolactin and/or prolactin mimetics is brought about by a slow release bolus.

33. A method as claimed in claim 1 wherein the sustained increase in circulating level of prolactin and/or prolactin mimetics is brought about by way of an implant.

34. A method as claimed by claim 29, wherein the reduction in circulating level of prolactin and/or prolactin mimetics is brought about by decreasing the photoperiod to which the animal is exposed.

35. A method as claimed in claim 29, wherein the decrease in circulating level of prolactin and/or prolactin mimetics is brought about by reducing or terminating the exogenous administration of prolactin or prolactin mimetics.

36. A method as claimed in claim 29, wherein the sustained increase is for a period of 3 to 18 days.

37. A method as claimed in claim 36, wherein the sustained increase is for a period of 3 to 15 days.

38. A method as claimed in claim 37, wherein the sustained prolactin increase is for a period of 3 to 9 days.

39. A method as claimed in claim 29, wherein the circulating level of prolactin is increased by 5 ng/ml to 800 ng/ml.

40. A method as claimed in claim 39, wherein the circulating level of prolactin is increased by 5 ng/ml to 200 ng/mg.

41. A method as claimed in claim 29, wherein the increase in the circulating level of prolactin is brought about by the incorporation into the animal's genome of an inducible recombinant nucleotide sequence encoding biologically active prolactin.

42. A method as claimed in claim 29, wherein the increase in the circulating level of prolactin is brought about by the incorporation into the animal's genome of a recombinant nucleotide sequence encoding a molecule which enhances endogenous prolactin activity.

43. A method as claimed in claim 41, wherein the recombinant nucleotide sequence is inserted into an inducible gene cassette under the control of a suitable promoter and/or enhancer sequence.

44. A method as claimed in claim 42, wherein the recombinant nucleotide sequence is inserted into an inducible gene cassette under the control of a suitable promoter and/or enhancer sequence.

45. A method as claimed in claim 43, wherein the promoter is mammary specific.

46. A method as claimed in claim 43, wherein the promoter is a milk protein.

47. A method as claimed in claim 43, wherein the promoter and/or enhancer sequence drives the transcription of the recombinant nucleotide sequence.

48. A method as claimed in claim 41, wherein the recombinant nucleotide sequence contains 3′ flanking DNA to stabilize an mRNA.

49. A method as claimed in claim 43, wherein the gene cassette contains downstream regulatory sequences.

50. A method as claimed in claim 1, wherein the modulation of prolactin receptors is achieved by the administration of antibodies.

51. An animal treated by the method as claimed in claim 1.

52. Animal products produced by an animal treated by the method as claimed in claim 1.
